(19) United States
(12) Patent Application Publication
(10) Pub. No.: US 2021/0308522 A1
(43) Pub. Date: Oct. 7, 2021

(54) SYSTEM AND METHOD FOR REAL-TIME ENVIRONMENTALLY NORMALIZED ENDURANCE ATHLETIC TRAINING AND COMPETITION

(71) Applicant: Jeffrey Booher, Southlake, TX (US)

(72) Inventor: Jeffrey Booher, Southlake, TX (US)

(73) Assignee: Predictive Fitness Inc., Southlake, TX (US)

(21) Appl. No.: 16/936,400

(22) Filed: Jul. 22, 2020

Related U.S. Application Data
(63) Continuation-in-part of application No. 13/475,850, filed on May 18, 2012.
(60) Provisional application No. 61/488,084, filed on May 19, 2011.
(60) Provisional application No. 61/646,765, filed on May 14, 2012.

(30) Foreign Application Priority Data
May 18, 2012 (US) PCT/US2012/038708

(51) Int. Cl. A63B 24/00; A63B 71/06

(52) U.S. Cl. CPC A63B 24/0062; A63B 24/0084; A63B 2230/06; A63B 2220/12; A63B 71/0622

(57) ABSTRACT

A near real-time GPS and computer-implemented system and method for measuring the physical capability of an athlete in an athletic endurance event and displaying, in near real-time, performance parameters after adjustment by at least one environmental normalization factor. The system and method performs the following steps in a specially programmed computer in near real-time: accepting near real-time data input from at least one sensor taken from the group of heart rate monitor for measuring an individual athlete's heart rate and a GPS device for determining distance traveled by the athlete in a time period, accepting environmental data correlated to the factor in near real-time, the data being accepted from at least one of a remote transmitter or a measurement device worn or carried by the athlete, computationally adjusting the athlete's measured actual performance by means of at least one environmental normalization factor taken from the group of temperature, humidity and elevation, wind, air density, and elevation change (hills), to compute an environmentally normalized performance value, dynamically recalculating said normalized performance values in near real-time based on additional environmental data accepted at subsequent times during said performance, and providing an indication in near real-time to the athlete during said performance regarding whether the current performance is within specified limits as compared to the normalized performance values for the individual athlete including adjustment by said normalized environmental factor to allow the athlete to take timely action during said performance to improve his or her performance. The system and method also enables virtual real-time events where environment normalization displays real-time progress/results for participants in different environments while they compete in the same event. The system and method normalizes the results from users participating at high altitude versus those a lower altitude (along with wind, temperature, elevation change, etc.) so that all participants effectively compete on a virtual “level playing field” during the event. Environment normalization can validate or certify real-time that performance data submitted during virtual racing events are valid based on the athletes actual environment normalized potential.

### RELATED APPLICATIONS

This application claims priority to U.S. provisional application Ser. No. 61/488,084, filed May 19, 2011; U.S. provisional application Ser. No. 61/646,765, filed May 14, 2012; PCT application PCT/US2012/038708, filed May 18, 2012; and pending U.S. Non-Provisional Application Ser. No. 13/475,850, filed May 18, 2011; the disclosure of which is hereby incorporated by reference herein for all purposes without disclaimer.

### TECHNICAL FIELD

This application is directed, in general, to a training method and system, and, more specifically, to a system and computer-implemented method for producing individually customized training programs for multi-discipline endurance athletic competitions, such as triathlons.

### BACKGROUND

Triathlons and other multi-discipline endurance athletic competitions having at least two distinct disciplines are an increasingly important aspect of athletics. One of the biggest challenges is to measure and describe an individual athlete's current performance abilities in each discipline in a meaningful way so that training can be focused and optimized for the best results and most efficient use of available training time.

This description for the athlete should be a way in which the athlete can easily measure progress; not only in overall race performance, but also in the various subsets of abilities that contribute to the athlete's overall race performance, i.e., a way in which the athlete can easily identify limiters. This can be a way that is specific to each discipline of the triathlon. This should ideally be a way that communicates, not only the athlete's athletic potential, but the athlete's ability to actualize that potential.

This description for the athlete should be a way in which the athlete can easily measure relative differences in performance ability for each discipline (swim, bike, and run), the potential for improvement in each discipline, and the impact of that improvement on overall results when competing in triathlons of various distances as the proportions of swim, bike, and run duration differ. For purposes of this application, the term “discipline” is used to mean a distinctly different mode of athletic endurance event or race (such as running vs. biking vs. swimming or pull-ups or push-ups) as opposed to different times or distances of the same mode of racing or endurance event (such as a 10K run vs. a 5K run).

For example, in a first approach to describe a triathlon performance level, the athlete could use a most recent race finish time. However, this naïve approach would not differentiate the athlete's performance by each discipline. Nor would this naïve system communicate how that time compared to a current ability or potential. Nor would it indicate the athlete's limiters, or be useful in prioritizing the athlete's training or setting the athlete's training focus.

One advantage of having a coach or coaches can be realizing a benefit from their ability to interpret training data based on their experience. This can be in addition to use of software adapted to analyze training files from heart rate monitors and power meters. One benefit conveyed by a coach is a coach's ability to measure progress looking at key indicators and then tweaking the athlete's training plan appropriately. However, disadvantageously, this can be a very unstructured process that differs for every coach. In general, triathlon coaches do not have systems for comparing race or training results from athlete to athlete (of various backgrounds, body compositions, ages, etc., making their “experience” and conclusions about effectiveness of training programs more or less guesswork based on general training theories. Triathlon coaches do not have metrics for relating relative differences in swim, bike, run abilities or thresholds for when or by how much training focus should be shifted.

One prior art approach for training runners dealt with a “running formula,” derived by a Mr. Jack Daniels and one of his athletes, which was a quantified measurement of athletic ability called a “VDOT.” A VDOT is essentially an athlete's running velocity at VO₂ₘₐₓ (their maximum oxygen uptake.) Daniels then created a chart that showed race times and training paces based on a runner's VDOTs. For more information on how he developed VDOT and what it means please refer to his book entitled “Daniel's Running Formula.” which can be experimentally derived VDOT values. The “formula” may also refer to an overall approach and philosophy on running.

VDOTs are useful as an effort to take into account both physiological capacity and potential for improvement in running only. However, because triathlon training involves three disciplines and distributing training focus and workload, it presents problems that are not present in standard training for stand-alone sports such as running. Therefore, there is a need for a triathlon training and racing system that addresses at least some of the concerns associated with conventional training programs for triathlons.

### SUMMARY OF THE INVENTION

In an embodiment of the invention, an apparatus for creating and printing or displaying to a user an individually customized multi-discipline training plan for improving the physical capability of an athlete in a multi-discipline athletic endurance event is provided. The apparatus comprises a specially programmed computer that generates a normalized performance value of an individual athlete for each of at least two different disciplines of a multi-discipline athletic endurance event and generates a training plan customized for the athlete correlated to achieving an incremental improvement in the normalized performance values for each of the disciplines within a particular period of time; and an output device operatively connected to the specially programmed computer for printing or displaying the training plan.

In another aspect, one of the disciplines is running.

In another aspect, one of the disciplines is swimming.

In another aspect, one of the disciplines is biking.

In another aspect, the disciplines are taken from the group of running, bicycling and swimming.

In another aspect, there are at least three disciplines.

In another aspect, the disciplines including running, bicycling and swimming.

In another aspect, the training plan is output to a user.

In another aspect, the normalized performance values are computed based upon input of the individual athlete's assessment values for each of the disciplines.

In another aspect, the specially programmed computer correlates the normalized performance values to expected competitive performance results for at least one subtype of each of the disciplines and the output device operatively connected to the specially programmed computer generates electronically or physically printed expected competitive performance results for at least one subtype of each of the disciplines correlated to the normalized performance values.

In another aspect, the specially programmed computer correlates the expected incremental improvement in the normalized performance values from following the training plan to expected competitive performance results for at least one subtype of each of the disciplines at a future time and the output device operatively connected to the specially programmed computer generates electronically or physically printed the expected competitive performance results for at least one subtype of each of the discipline.

In another aspect, the training plan includes a set of exercises having at least volume and intensity instructions individually tailored based on data entered specific to the individual athlete, the volume and intensity instructions being optimized to reduce the differences between the individual athlete's normalized performance values for each of the disciplines.

In another aspect, the training plan includes a set of exercises having at least volume and intensity instructions individually tailored based on data entered specific to the individual athlete, the volume and intensity instructions being optimized to improve the athlete's expected overall score for an intended multi-discipline competition at a future date.

In another aspect, the apparatus further comprises means for inputting a parameter correlating to an athlete's available training time between the present time and a future date; and means for determining by means of the specially programmed computer a limiting parameter correlative of the athlete's physical capability to safely perform a particular workload during a particular increment of the available training time; and the specially programmed computer generates a training plan designed to provide the individual athlete with an amount of incremental improvement in the normalized performance values without exceeding the limiting parameter correlative of the athlete's physical capability to safely perform a particular workload during a particular increment of the available training time.

In another aspect, the apparatus further comprises means for determining the maximum amount of incremental improvement in at least one of the normalized performance values that can be expected to be achieved by the individual athlete in the available training time without exceeding the limiting parameter correlative of the athlete's physical capability to safely perform a particular workload during a particular increment of the available training time and the specially programmed computer generates a training plan designed to provide the individual athlete with the maximum amount of the incremental improvement.

In another aspect, the apparatus further comprises means for determining the maximum amount of total incremental improvement in the normalized performance values for the disciplines that can be expected to be achieved by the individual athlete in the available training time without exceeding the limiting parameter correlative of the athlete's physical capability to safely perform a particular workload during a particular increment of the available training time and the specially programmed computer generates a training plan designed to provide the individual athlete with the maximum amount of total incremental improvement.

In another aspect, the apparatus further comprises means for determining the maximum amount of total incremental improvement in a competition score for a target competition on the future date that can be expected to be achieved by the individual athlete in the available training time without exceeding the limiting parameter correlative of the athlete's physical capability to safely perform a particular workload during a particular increment of the available training time and the specially programmed computer generates a training plan designed to provide the individual athlete with the maximum amount of the incremental improvement in the competition score.

In another aspect, the apparatus further comprises means for inputting a desired competition score for a target competition on the future date; means for determining the maximum expected amount of total incremental improvement in each of the normalized performance values that can be expected to be achieved by the individual athlete in the available training time without exceeding the limiting parameter correlative of the athlete's physical capability to safely perform a particular workload during a particular increment of the available training time and means for determining if the at least one combination of normalized performance values will permit the athlete to achieve the desired competition score in the target competition on the future date without exceeding the maximum expected amount of total incremental improvement and, if so, generating by means of a specially programmed computer a training plan designed to provide the individual athlete with the desired competition score.

In another aspect, the limiting parameter is taken from the group of volume, workload, workload stress.

In another aspect, the limiting parameter is workload stress.

In another aspect, the workload stress is a function of workload intensity and time at the intensity.

In another aspect, the workload stress is further correlated to a factor that increases during the time that an exercise is performed.

In another aspect, the apparatus further comprises means for determining an athlete's current normalized performance values after a portion of the particular period of time has elapsed, and means for regenerating a new training plan customized for the athlete correlated to achieving an incremental improvement in the normalized performance values for each of the disciplines in the remaining amount of the particular period of time.

In another aspect, the apparatus further comprises a memory for storing data input by a user relating to the athletic ability of an athlete, the data including at least a first assessment, the training plan followed by the athlete, and a second assessment following same training plan, means for combining the data input by the user to other data correlating training plans and expected changes in assessments during a period of time that are applicable to a large number of athletes on a statistical basis; and means for modifying one or more factors correlating to normalized performance values based upon the data input by the user along with the other data.

In another aspect, the training plans include information regarding a plurality of exercise sessions.

In another aspect, each of the exercise sessions includes information specifying, for at least one discipline, one or more of the number of repetitions, duration of a session, intensity of a session, and the number of sets of repetitions.

In another aspect, the apparatus further comprises means for determining an expected competition score at least one discipline at a time in the future based upon the athlete's current normalized performance values in the disciplines.

In another aspect, the apparatus further comprises means for determining an expected competition score in at least one discipline at a time in the future based upon the athlete's expected normalized performance values in the disciplines after performance of the training plan during the particular period of time.

In another aspect, the apparatus further comprises means for inputting an individual athlete's desired total score for a target competition on a target date in the future; . . . means for determining by means of a specially programmed computer the total workload required to achieve the desired total score of the target competition; and means for generating by means of a specially programed computer an individually customized training plan for the individual athlete to achieve the desired total score for the target competition on the target date in the future without exceeding permissible maxima during all or a portion of the training period prior to the target date of one or more of volume, workload, or workload stress applicable to the individual athlete.

In another aspect, one of the disciplines is bicycling and the maxima of volume, workload, or workload stress applicable to the individual athlete are functions at least in part of (1) power-to-total-weight ratio with bike weight and (2) BMI.

In another aspect, one of the disciplines is running and wherein the apparatus further comprises means for determining by means of a specially programed computer a pace to weight ratio for the running discipline; and the maxima of volume, workload, or workload stress applicable to the individual athlete are determined at least in part by the pace to weight ratio.

In another embodiment, a computer-implemented method for creating and printing or displaying to a user individually customized multi-discipline training plan for improving the physical capability of an athlete in a multi-discipline athletic endurance event is provided, comprising the steps of: computing a normalized performance value of an individual athlete for each of at least two different disciplines of a multi-discipline athletic endurance event; and generating an electronically or physically printed training plan customized for the athlete correlated to achieving an incremental improvement in the normalized performance values for each of the disciplines within a particular period of time.

In another aspect, one of the disciplines is running.

In another aspect, one of the disciplines is swimming.

In another aspect, one of the disciplines is biking.

In another aspect, the disciplines are taken from the group of running, bicycling and swimming.

In another aspect, there are at least three disciplines.

In another aspect, the disciplines including running, bicycling and swimming.

In another aspect, the training plan is output to a user.

In another aspect, the normalized performance values are computed based upon input of the individual athlete's assessment values for each of the disciplines.

In another aspect, the specially programmed computer correlates the normalized performance values to expected competitive performance results for at least one subtype of each of the disciplines and the output device operatively connected to the specially programmed computer generates electronically or physically printed expected competitive performance results for at least one subtype of each of the disciplines correlated to the normalized performance values.

In another aspect, the specially programmed computer correlates the expected incremental improvement in the normalized performance values from following the training plan to expected competitive performance results for at least one subtype of each of the disciplines at a future time and the output device operatively connected to the specially computer generates electronically or physically printed the expected competitive performance results for at least one subtype of each of the discipline.

In another aspect, the training plan includes a set of exercises having at least volume and intensity instructions individually tailored based on data entered specific to the individual athlete, the volume and intensity instructions being optimized to reduce the differences between the individual athlete's normalized performance values for each of the disciplines.

In another aspect, the training plan includes a set of exercises having at least volume and intensity instructions individually tailored based on data entered specific to the individual athlete, the volume and intensity instructions being optimized to improve the athlete's expected overall score for an intended multi-discipline competition at a future date.

In another aspect, the method further comprises inputting a parameter correlating to an athlete's available training time between the present time and a future date; determining by means of the specially programmed computer a limiting parameter correlative of the athlete's physical capability to safely perform a particular workload during a particular increment of the available training time, and the specially programmed computer generates a training plan designed to provide the individual athlete with an amount of incremental improvement in the normalized performance values without exceeding the limiting parameter correlative of the athlete's physical capability to safely perform a particular workload during a particular increment of the available training time.

In another aspect, the method further comprises determining the maximum amount of incremental improvement in at least one of the normalized performance values that can be expected to be achieved by the individual athlete in the available training time without exceeding the limiting parameter correlative of the athlete's physical capability to safely perform a particular workload during a particular increment of the available training time and The specially programed computer generates a training plan designed to provide the individual athlete with the maximum amount of the incremental improvement.

In another aspect, the method further comprises determining the maximum amount of total incremental improvement in the normalized performance values that can be expected to be achieved by the individual athlete in the available training time without exceeding the limiting parameter correlative of the athlete's physical capability to safely perform a particular workload during a particular increment of the available training time and the specially programmed computer generates a training plan designed to provide the individual athlete with the maximum amount of total incremental improvement.

In another aspect, the method further comprises determining the maximum amount of total incremental improvement in a competition score for a target competition on the future date that can be expected to be achieved by the individual athlete in the available training time without exceeding the limiting parameter correlative of the athlete's physical capability to safely perform a particular workload during a particular increment of the available training time and the specially programmed computer generates a training plan designed to provide the individual athlete with the maximum amount of the incremental improvement in the competition score.

In another aspect, the method further comprises inputting a desired competition score for a target competition on the future date; determining the maximum expected amount of total incremental improvement in each of the normalized performance values that can be expected to be achieved by the individual athlete in the available training time without exceeding the limiting parameter correlative of the athlete's physical capability to safely perform a particular workload during a particular increment of the available training time and determining if the at least one combination of normalized performance values will permit the athlete to achieve the desired competition score in the target competition on the future date without exceeding the maximum expected amount of total incremental improvement and, if so, generating by means of a specially programmed computer a training plan designed to provide the individual athlete with the desired competition score.

In another aspect of the method, the limiting parameter is taken from the group of volume, workload, workload stress.

In another aspect of the method, the limiting parameter is workload stress.

In another aspect of the method, the workload stress is a function of workload intensity and time at the intensity.

In another aspect of the method, the workload stress for the athlete is a function of the athlete's workload capacity.

In another aspect of the method, the workload stress is further correlated to a factor that increases during the time that an exercise is performed.

In another aspect, the method further comprises determining an athlete's current normalized performance values after a portion of the particular period of time has elapsed, and regenerating a new training plan customized for the athlete correlated to achieving an incremental improvement in the normalized performance values for each of the disciplines in the remaining amount of the particular period of time.

In another aspect, the method further comprises storing data input by a user relating to the athletic ability of an athlete, the data including at least a first assessment, the training plan followed by the athlete, and a second assessment after following said training plan, combining the data input by the user to other data correlating training plans and expected changes in assessments during a period of time that are applicable to a large number of athletes on a statistical basis; and modifying one or more factors correlating to normalized performance values based upon the data input by the user along with the other data.

In another aspect of the method, the training plans include information regarding a plurality of exercise sessions.

In another aspect of the method, each of the exercise sessions includes information specifying, for at least one discipline, one or more of the number of repetitions, duration of a session, intensity of a session, and the number of sets of repetitions.

In another aspect, the method further comprises determining an expected competition score at least one discipline at a time in the future based upon the athlete's current normalized performance values.

In another aspect, the method further comprises determining an expected competition score in at least one discipline at a time in the future based upon the athlete's expected normalized performance values after performance of the training plan during the particular period of time.

In another aspect, the method further comprises inputting an individual athlete's desired total score for a target competition on a target date in the future; determining by means of a specially programmed computer the total workload required to achieve the desired total score of the target competition; and generating by means of a specially programmed computer an individually customized training plan for the individual athlete to achieve the desired total score for the target competition on the target date in the future without exceeding permissible maxima during all or a portion of the training period prior to the target date of one or more of volume, workload, or workload stress applicable to the individual athlete.

In another aspect of the method, one of the disciplines is bicycling and the maxima of volume, workload, or workload stress applicable to the individual athlete are athlete are functions at least in part of (1) power-to-total-weight ratio with bike weight and (2) BMI.

In another aspect of the method, one of the disciplines is running and wherein the apparatus further comprises means for determining by means of a specially programed computer a pace to weight ratio for the running discipline; and;

In another aspect of the method, the maxima of volume, workload, or workload stress applicable to the individual athlete are determined at least in part by the pace to weight ratio.

In another embodiment of the invention, a computer-implemented method for creating and printing or displaying to a user individually customized multi-discipline training plan for improving the physical capability of an athlete in a multi-discipline athletic endurance competition is provided, comprising storing a set of reference performance values correlated to average finishing times for a plurality of well-trained athletes measured when the athletes are performing at substantially maximum effort during one or more of the endurance disciplines that comprise the competition, the scales of the reference performance values for each discipline being normalized to each other so that substantially the same relative athletic performance ability will be represented by the same reference performance values; computing, using a specially programmed computer, a set of individual performance values correlated to an individual athlete's performance ability in each of the endurance disciplines, the scales of the individual performance values for each discipline being normalized to each other so that substantially the same relative performance ability of the individual athlete in each discipline as compared to the reference performance value in each discipline will be represented by the same individual performance values; and computing an expected competitive performance result in each of the disciplines as a function of the individual performance values and enabling printing and/or displaying the values to a user to guide the athlete in improving his/her physical capability as measured by the individual performance values.

In another aspect, the method further comprises computing a set of competition performance results in each of the disciplines that is expected to correlate to a set of the individual performance values and enabling printing and/or displaying the performance values and results to a user to guide the athlete in improving his/her physical capability as measured by the individual performance values to improve the expected competition performance results.

In another aspect, the method further comprises selecting, using a specially programmed computer, a set of workouts to be performed over a period of time, each of the workouts having a degree of athletic effort measured by at least volume and intensity, the workouts being correlated to an increment of increase of the individual performance values over the period of time.

In another aspect, the method further comprises selecting the number, volume and intensity of the workouts to maximize a total expected increment of increase of competition performance for a given number of hours of training time devoted to the workouts during the period of time.

In another aspect of the method, the number, volume and intensity of the workouts are selected to maximize a total expected increment of increase of competition performance for a given number of hours of training time devoted to the workouts during the period of time.

The computer system can develop training plans based on each of these performance values. For convenience, examples of these performance values used in connection with triathlons (which have running, biking and swimming disciplines) will be given. Such performance values for triathlons will generally be referred to herein as “TriDots.” The calculations consist of using the TriDots and other athlete data to look up values, thresholds, ratios, and other training parameters that are correlated to competitive performance results for each discipline, based upon data applicable to average performance by well-trained athletes in each discipline. Thus, TriDots and other athlete data is used to determine how many quality sessions (high-intensity) sessions an athlete should do in a week for each discipline, how much of a particular intensity an athlete should do per week and per quality session, what multi-week, weekly, and single-session workloads should the program reach prior to the race, based on the projected workload required to complete the race at the anticipated intensity level, to achieve an expected competitive result (i.e., an expected elapsed time for the race).

The computer system can compare the athlete's swimming, biking and running TriDots and show the athlete's relative performance level in each discipline, to identify and improve weak disciplines. For example, a TriDot profile of “40-42-35” (swim-bike-run) as calculated by the calculator 130 would indicate that a triathlete's run ability is significantly lower than the triathlete's swim and bike ability. Knowing this and using other athlete data, computer system can select workouts that focus the triathlete's training more on improving the athlete's run.

The computer system can also compare the athlete's swimming, biking and running TriDots and show the athlete's relative performance level in each discipline for purposes of optimizing training effort to maximize overall competitive performance during a race (which is measured by the total elapsed time for each of the disciplines, together). For example, depending upon relative distances for each of the running, biking and swimming events, despite a significant variation of TriDot values for each event, where the number of hours of total training prior to the time of a future race is limited (as is often the case), the computer system can select a set of workouts that will result in the greatest decrease in overall time for a further race that can be achieved by that athlete prior to the time of the race, using the available hours of training time, even if those workouts tend to improve TriDots for the different events unevenly.

### BRIEF DESCRIPTION OF THE DRAWINGS

Reference is now made to the following descriptions taken in conjunction with the accompanying drawings, in which:

FIG. 1 illustrates a computerized system for generating triathlon training and racing programs;

FIGS. 2A-2E illustrate an exemplary individually customized training plan (also occasionally referred to herein synonymously as a “training program”) computer-generated by an embodiment of the invention, with FIG. 2A showing a summary training phase profile, FIG. 2B showing training notes, FIG. 2C showing training paces, FIG. 2D showing successive weeks 1-12 of a training plan;

FIGS. 3A-3B illustrate interfaces for entering data into the computerized system of FIG. 1;

FIG. 3C depicts an embodiment of a user interface where the athlete enters their training request indicating the event type they're training for as well as their current training volume and long sessions by discipline;

FIGS. 4A-4H illustrate TriDot charts for quantifying performance ability in the run, swim and bike disciplines, showing relative differences between disciplines, and determining training intensities; of which:

FIG. 4A is a chart showing Stand-Alone Race Ability and Of-the-Bike Race Ability (3 sheets);

FIG. 4B is a chart showing Run TriDots at various rep paces (3 sheets);

FIG. 4C is a chart showing Run TriDots at various intervals paces (3 sheets);

FIG. 4D is a chart showing Run TriDots Threshold Pace and Tempo at various distances and tempos (3 sheets);

FIG. 4E is a chart showing Run TriDots Marathon and Easy (3 sheets) at various distances (3 sheets);

FIG. 4F is a chart showing Bike TriDots Time Trial, Threshold and Bike Split at Should Pace at various distances and speeds (3 sheets);

FIG. 4G is a chart showing Swim TriDots Time Trial, Threshold and Swim Split at Should Pace at various distances (3 sheets); and

FIG. 4H is a chart showing Race-Distance Progression Chart, Swim, Bike, Total Time at various distances (4 sheets).

FIG. 5 illustrates a method for generating a training program employing the TriDot System;

FIG. 6 includes representative source code that can be employed by the computerized system of FIG. 1 for generating a training program for a triathlon;

FIG. 7 is a table which describes how weekly increases are calculated;

FIG. 8 is a schematic showing an embodiment of the system of the invention depicting a specially programmed local computer to produce the individually customized training plans for the user, including user interfaces and permitting display and/or printing of individualized training plans.

FIG. 9 is a block diagram showing main components of a central server system acting as a website in communication with users over the internet, the central server system producing the individually customized training plans for the users and where webpages encompassing user interfaces and permitting display and/or printing of individualized training plans are transmitted over the internet to the users' computer terminals; and

FIG. 10 is a schematic showing an embodiment of the system of the invention where the users can access a central computer center over the internet where data is stored and processed to produce the individually customized training plans for the users and where webpages encompassing user interfaces and permitting display and/or printing of individualized training plans are transmitted to the users' computer terminals.

FIG. 11 is a schematic showing real-time inputs and outputs to and from an environmental normalization module.

### DETAILED DESCRIPTION

The numerous innovative teachings of the present application will be described with particular reference to presently preferred embodiments.

For simplicity and clarity of illustration, the drawing figures illustrate the general manner of construction, and description and details of well-known features and techniques may be omitted to avoid unnecessarily obscuring the invention. Additionally, elements in the drawing figures are not necessarily drawn to scale, some areas or elements may be expanded to help improve understanding of embodiments of the present disclosure.

The terms “first,” “second,” “third,” “fourth,” and the like in the description and the claims, if any, may be used for distinguishing between similar elements and not necessarily for describing a particular sequential or chronological order unless otherwise stated. It is to be understood that the terms so used are interchangeable. Furthermore, the terms “comprise,” “include,” “have,” and any variations thereof, are intended to cover non-exclusive inclusions, such that a process, method, article, apparatus, or composition that comprises a list of elements is not necessarily limited to those element, but may include other elements not expressly listed or inherent to such process, method, article, apparatus, or composition.

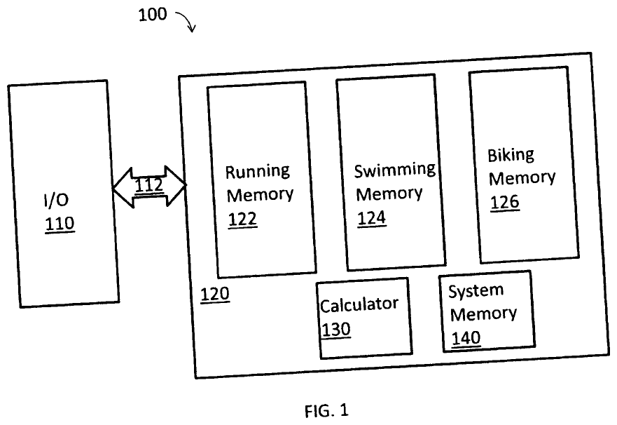

Turning now to FIG. 1, illustrated is a computerized system 100 for computing and producing an individually customized triathlon training program for a particular athlete. An input/output (I/O) device 110 is coupled over a bus 112 to a workstation 120. The workstation 120 has a running memory 122, a swimming memory 124, and a biking memory 126. These are variously coupled to a specially programmed processor for computing the various parameters (also referred to as a calculator herein) 130 and a system memory 140, which is in turn coupled to convey processed information over the bus 112. The system memory 140 can store global profile information about the particular multi-discipline athlete relevant to training (such as name, date of birth, gender, etc.).

Please note that the calculator 130 can be configured in hardware or firmware, and can be configured to run software to perform various aspects of the invention of the present Application. The calculator 130 can also further employ the TriDot values to generate a triathlon training program, as will be described below.

Generally, the present application is directed towards using measurements of Functional Threshold and Velocity at VO_{2Max} as applied in the context of a triathlon. The term Functional Threshold is especially applicable to endurance sports as it is a measure of an athlete's ability to perform work for a sustained period of time, generally an hour. For ease of explanation, this measure of Functional Threshold will be described as a “TriDot,” although this may also be referred to as a “triathlon” value or “normalized performance” value.

In one embodiment, the calculation of an athlete's TriDots begins with an assessment or time trial for each of the disciplines (swim, bike, and run). Trained athletes conduct assessments and record completion times for set distances or distances covered in a set time. These values are fed into the system 100 and a TriDot value is computed in the manner described herein.

TriDots are generally used to prescribe training intensities and race abilities based on the athlete's functional threshold. Modified assessments are available for beginner (untrained) athletes who are not yet capable of performing at a meaningful intensity for an hour, thus cannot technically test for a functional threshold. The results from these modified assessments are also fed into the system 100 to compute TriDot values. In this way, the system 100 can prescribe meaningful training intensities that will produce the desired training response (physiological adaptation) as if the athlete were able to perform a true functional threshold test.

Generally, the computerized system 100 can measure fitness for beginners (e.g., below VDOT 30). The computerized system 100 can use TriDots to prescribe power, pace, and heart-rate zones for various training intensities. There can be an exclusion of repetition and interval distances on a TriDot specific basis for distances that are too long or too short to be effective (i.e. A low TriDot runner would not typically do a 500 m repetition as it would take them too long to complete this distance to be effective as a “repetition intensity” effort).

In another embodiment, the system 100 can support a split pace for threshold, tempo, marathon-pace, and easy runs to allow athletes to monitor pace more often than every mile, and without a GPS. The system 100 can also be used to calculate projected bike splits and relative optimal intensities for overall effort during the bike leg of an event. It can also calculate projected off-the-bike run splits and pacing for all various triathlon distances. Bike and run pacing can be critical to overall performance as using heart rate and/or rating of perceived exertion alone as measures of intensity and endurance can be extremely misleading to the athlete during the event.

The TriDot assessment can be based on 5-kilometer and 10-kilometer time trials, as well as 12-minute time trial protocols for beginners. Also, all of the above can be done with swimming and biking, in addition to the run training (assessments, training paces, and race pacing guidance, for example).

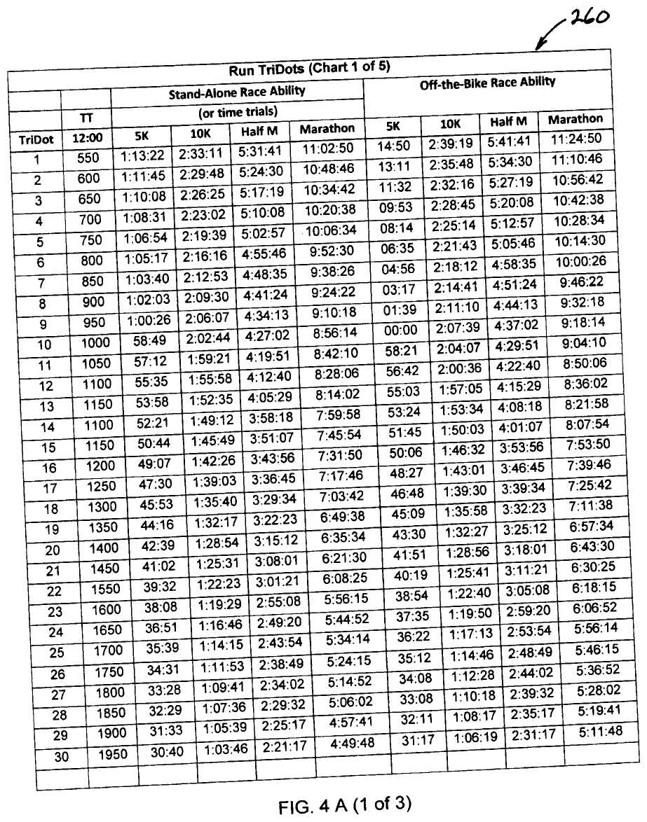

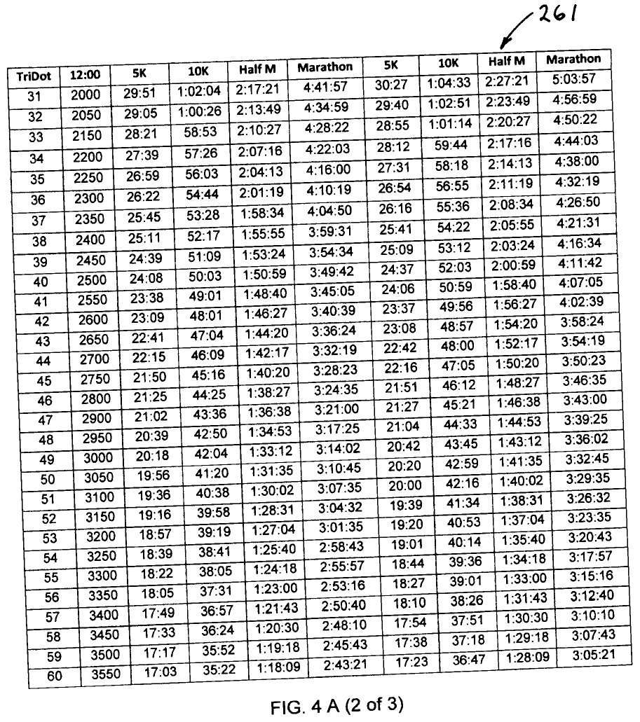

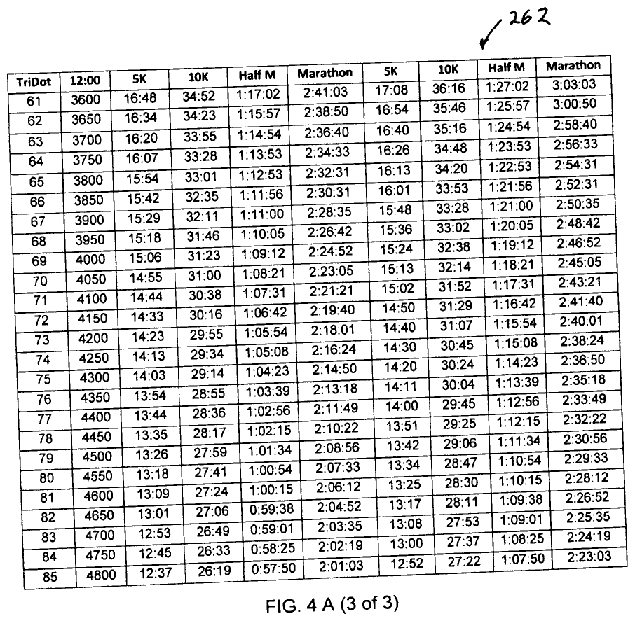

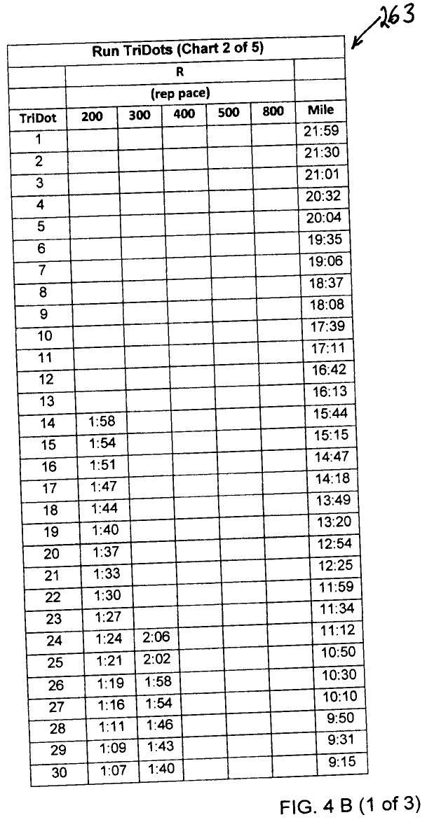

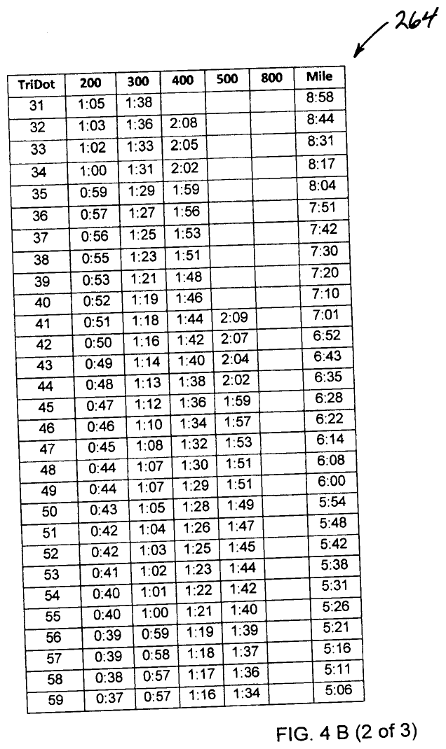

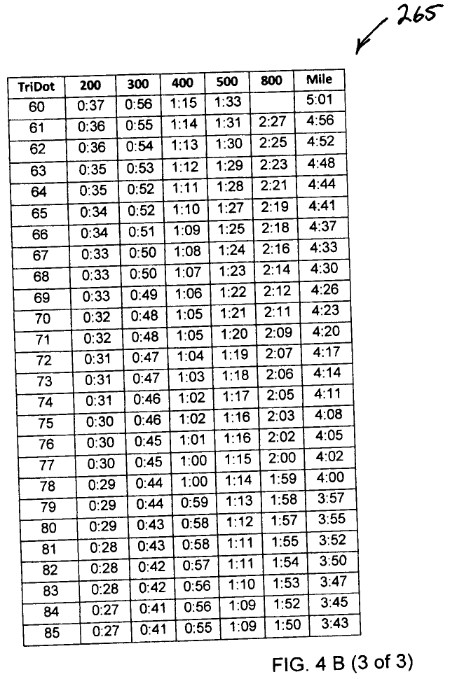

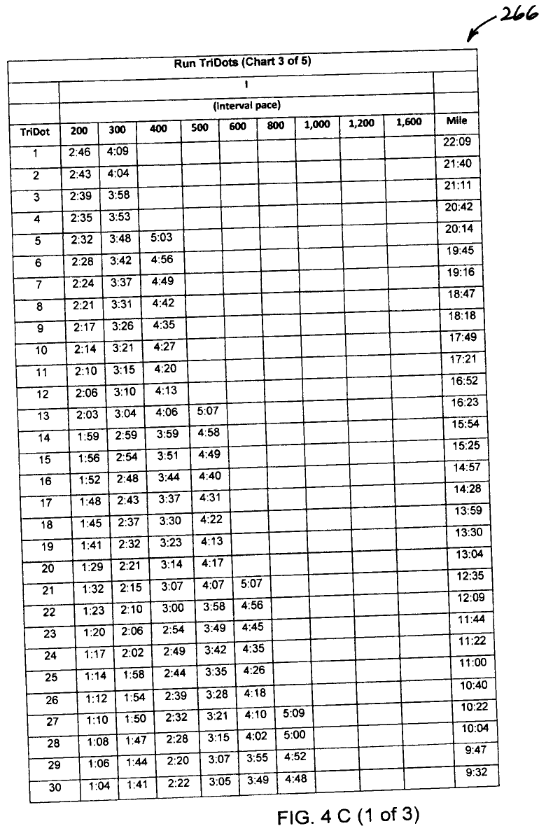

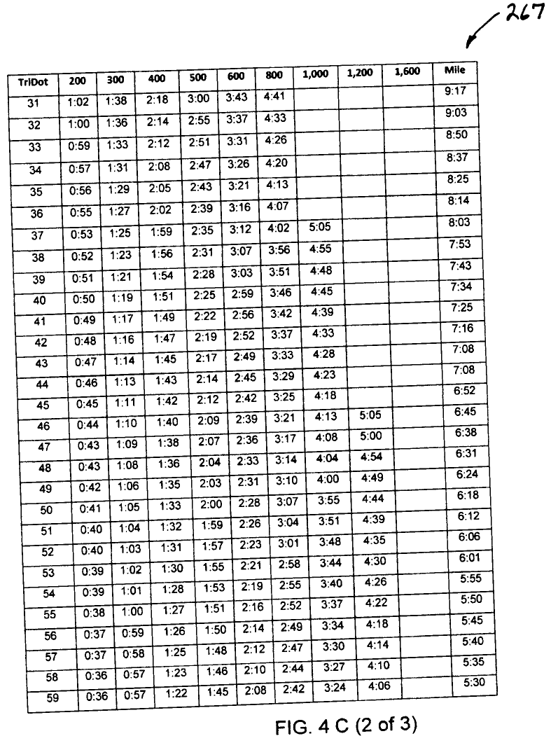

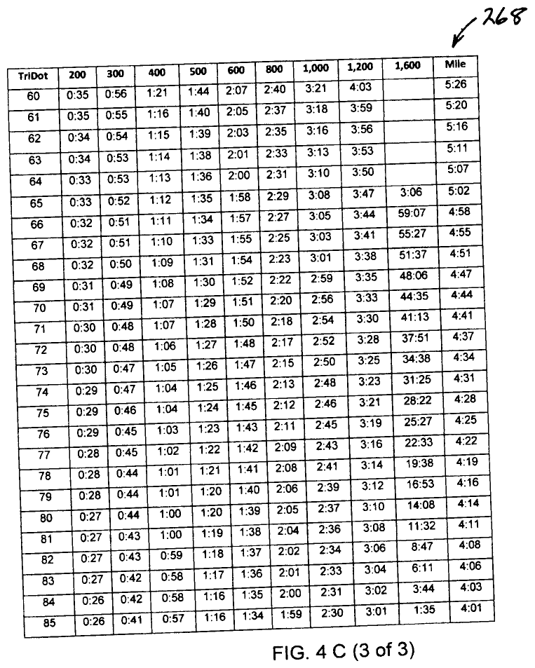

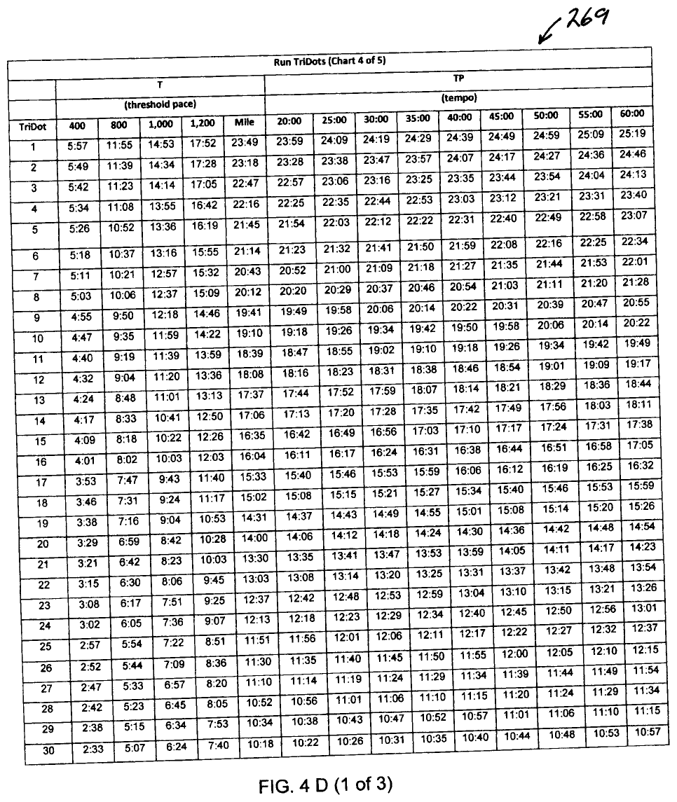

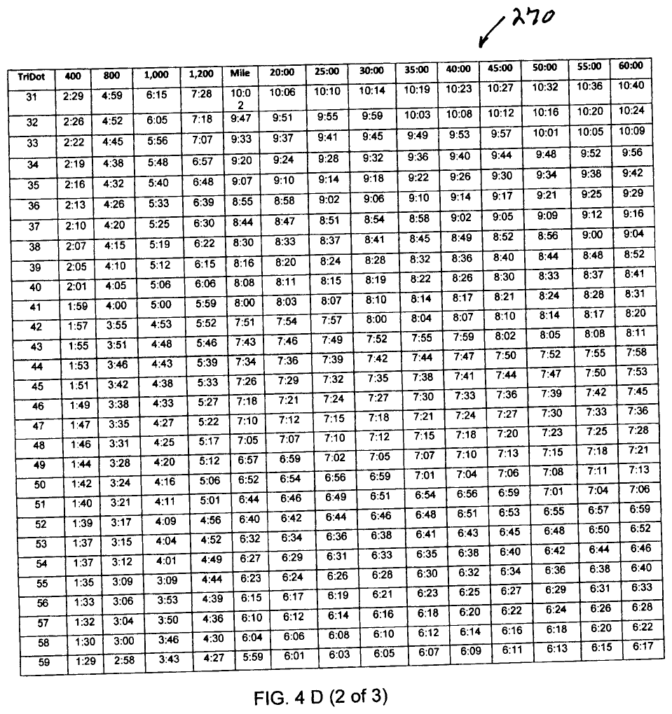

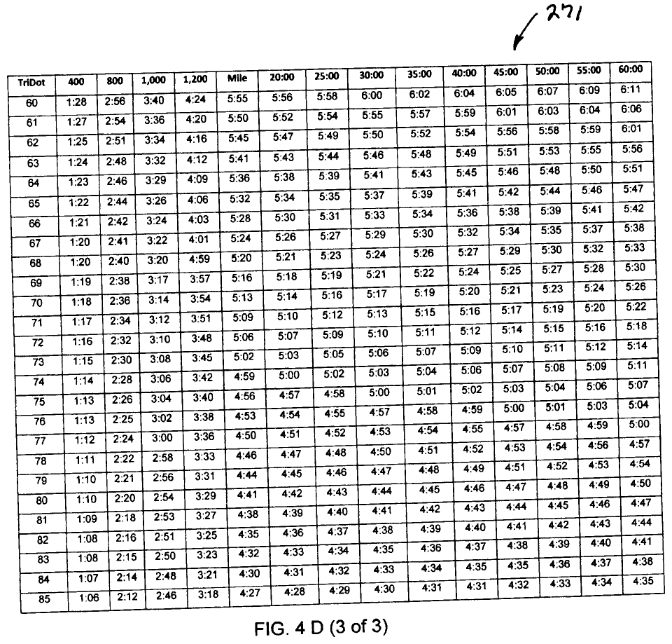

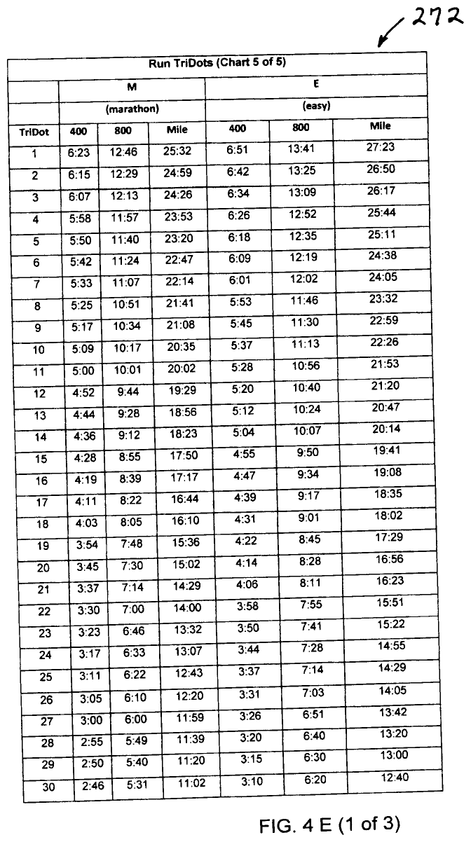

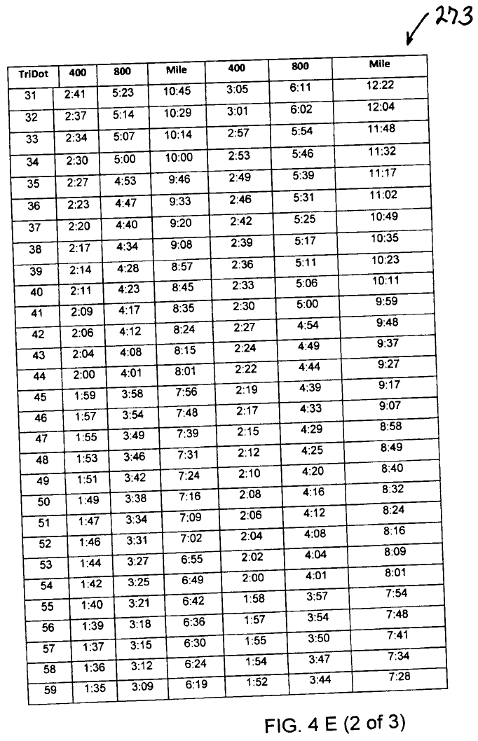

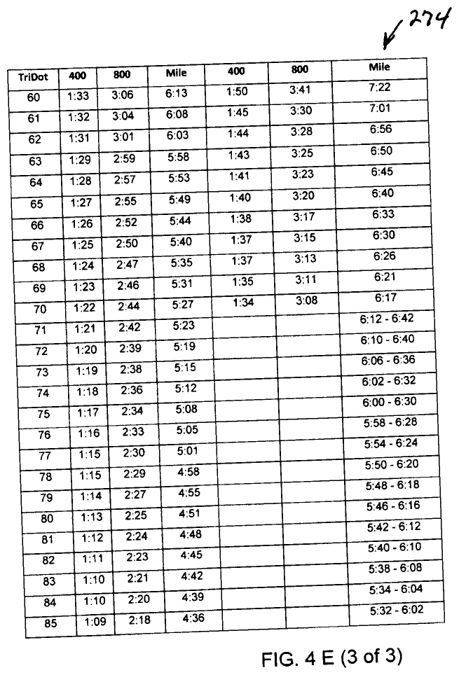

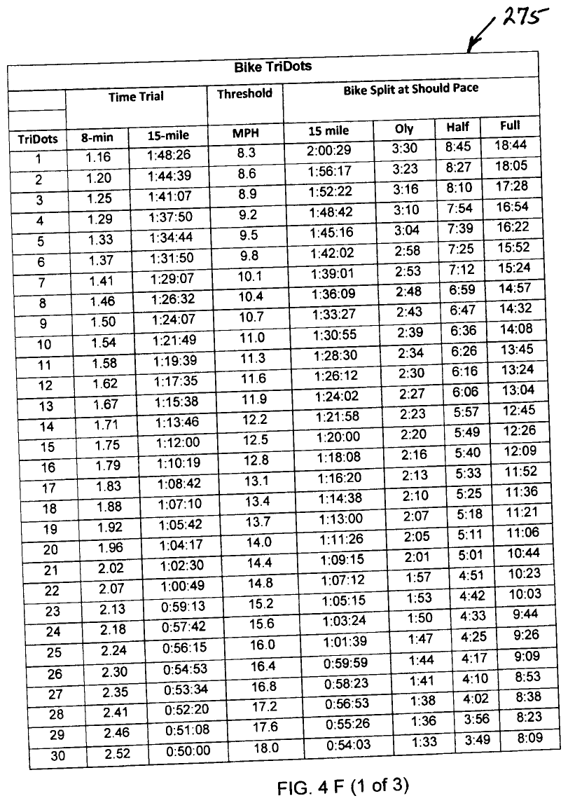

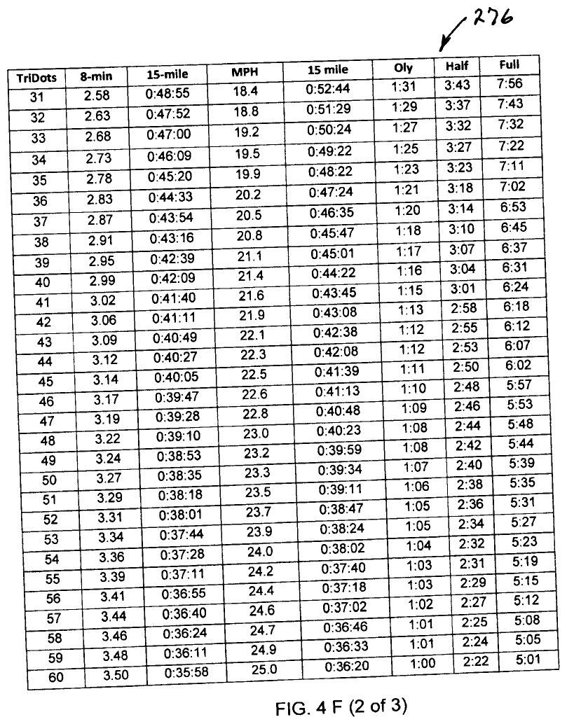

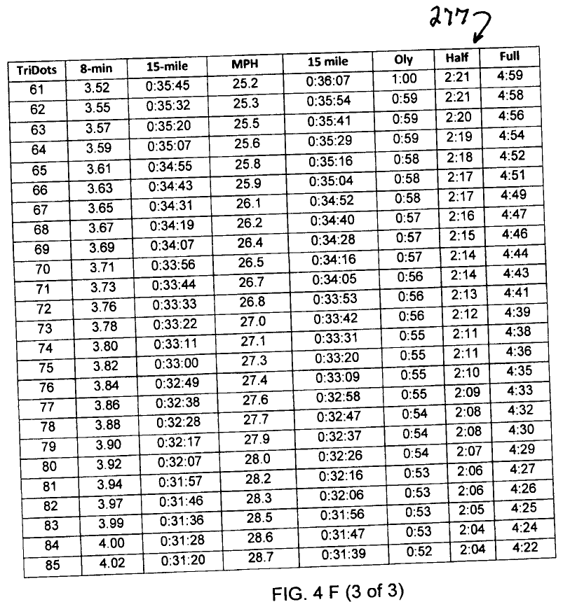

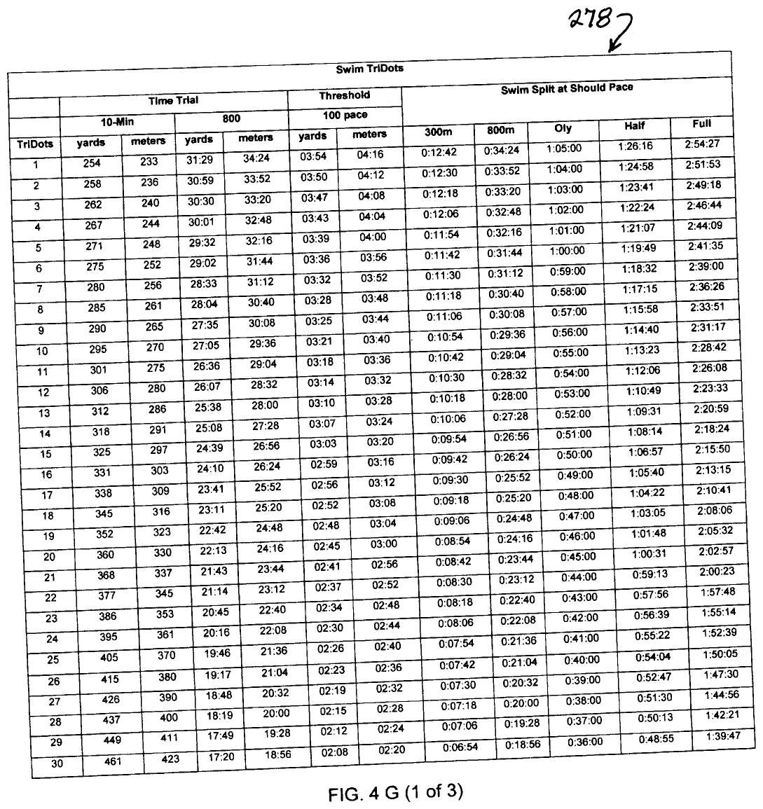

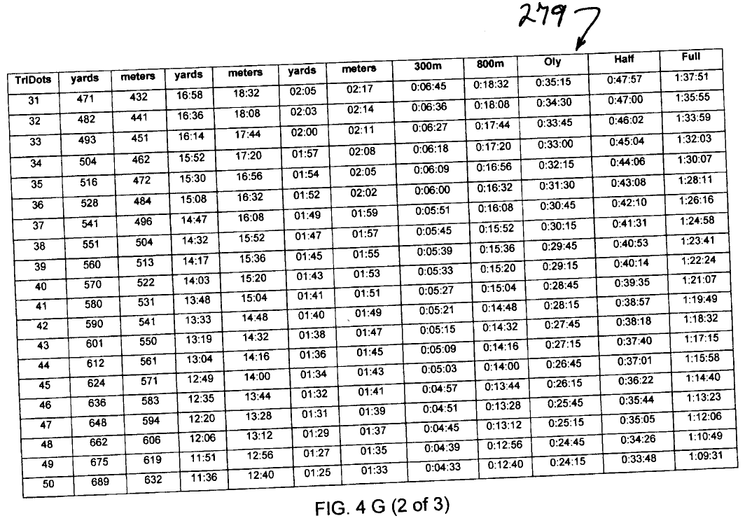

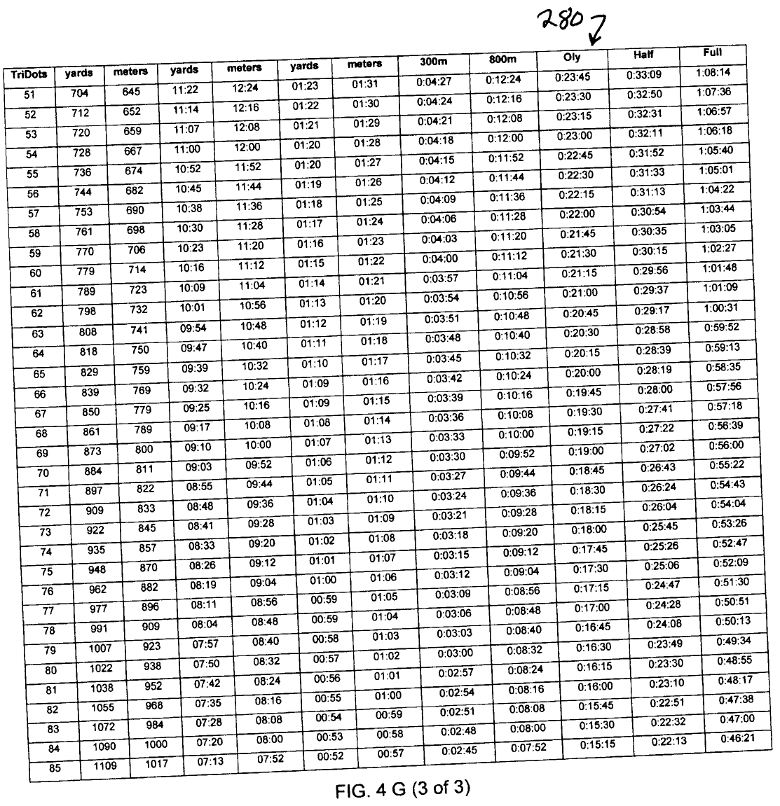

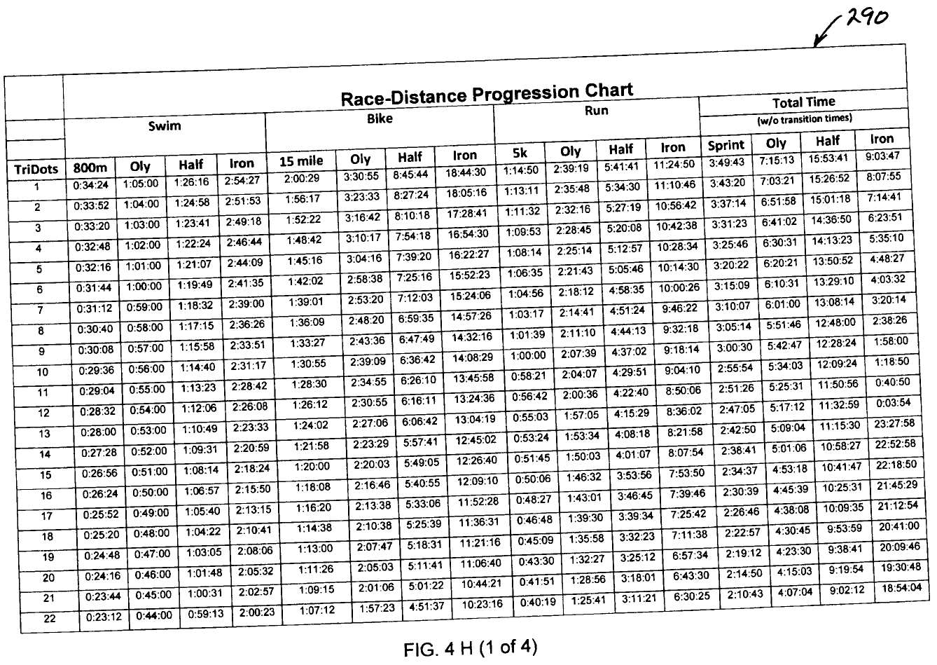

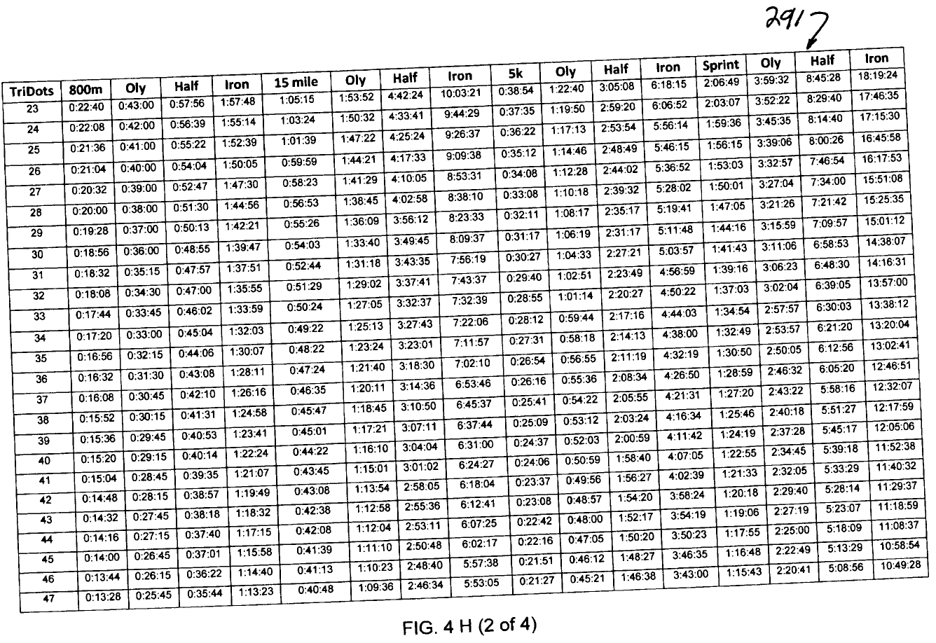

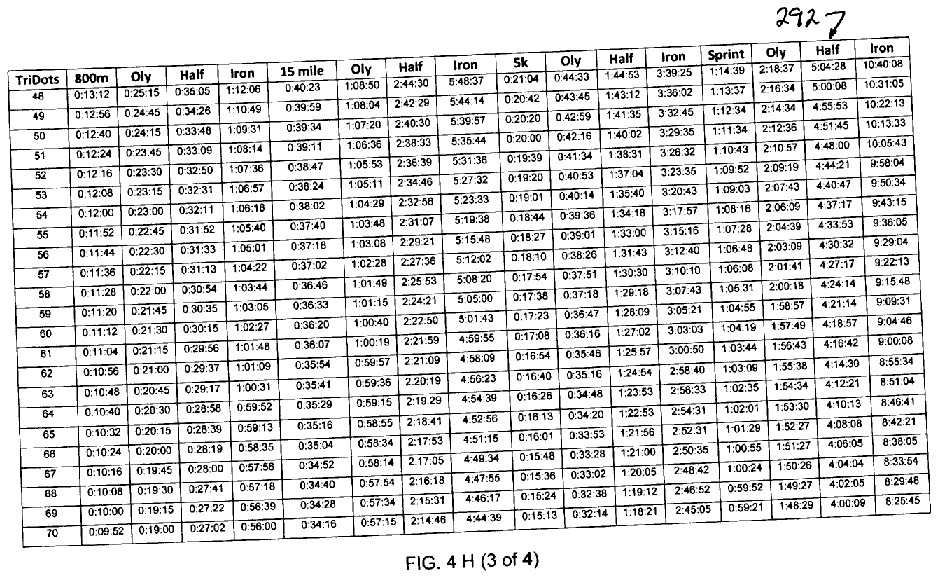

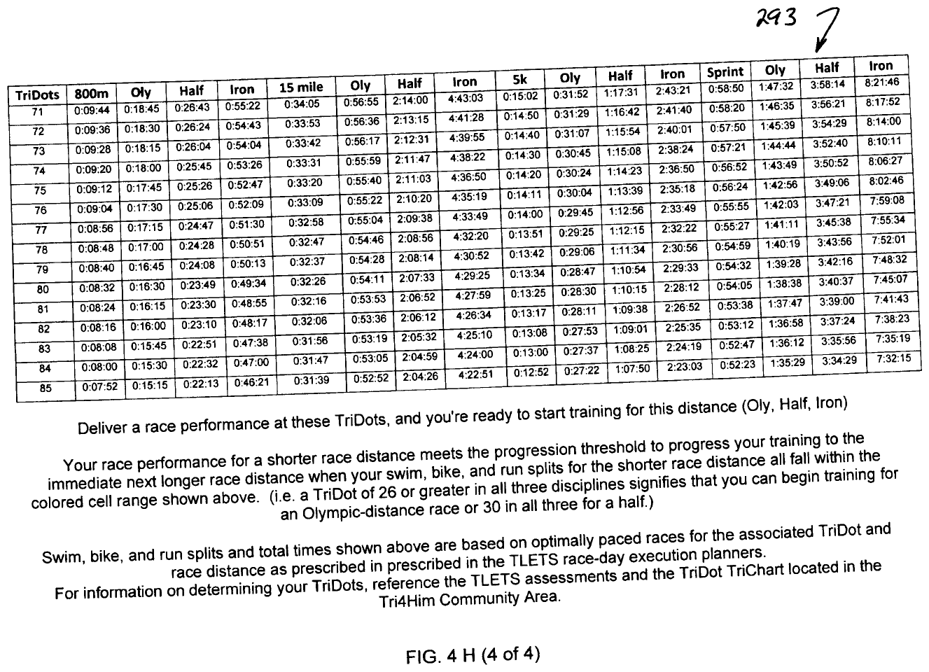

In one embodiment, triathletes can perform and quickly look up their performance on a TriDot chart manually to determine their swim, bike, and run TriDot, such as illustrated in FIGS. 4A to 4C.

With the performance measures as discussed above, the calculator 130 can calculate TriDot values. The calculator searches the applicable assessment table for the athlete's assessment time or distance and returns the associated TriDot value.

In one embodiment, rather than referring to a spreadsheet such as in FIG. 4A and 4B, “Tri-Charts”), triathletes simply enter their most recent time trial result through the 110 interface 110 and into the running memory 122, the swimming memory 124, and the biking memory 126. The calculator 130 then determines the TriDot for each of these values, and then stores this in the system memory 140. The system memory 140 then displays it on the I/O 110. The I/O 110 can also be configured to accept values directly from triathlete sensors (such as heart rate values from a wireless heart rate monitor and/or clocked time and distance measured by a GPS, for instance.)

In one embodiment, these values are displayed in an easy to read table on the I/O interface 110 and also in the margin of each week's training schedule. The entire training phase and each week's training schedule can also be generated by the calculator 130. The system compares TriDot values for the swim, bike, and run and evaluates any differences against a set of thresholds based on the athlete's race distance, background, training volume, etc. And appropriately determines the proper training volume (total and by discipline), optimal intensity mix (total and by discipline), and discipline volume and workload allocation to produce fitness gains that will translate to the greatest overall time improvement on race day. For example, a 10% improvement in swim time may not be as beneficial as a 10% improvement in bike time if the swim is 15% of a race and the bike is 50% of the race. The more advanced an athlete's skill is, the longer it will take to realize the same amount of additional fitness gains.

The system 100 prescribes training to produce gains in the areas of greatest potential. Some fitness gains cannot be realized until athletes have progressed through specific developmental stages that take certain periods of time engaged in consistent training. The system 100 recognizes these stages and develops other areas until these developmental stages are achieved.

In one embodiment, the calculator 130 also can perform conversions for athletes who train on treadmills, letting the triathlete know what setting to use to approximate the same effort on the road for each training intensity prescribed in a training plan. This is especially valuable in that it allows deliberate training in a controlled environment. Effort durations, intensities, and rest periods can be executed as prescribed in training plan without negative impact of terrain, traffic, and climate. Training sessions such as “hill repeats” can much more effectively be done on a treadmill. Optimal inclines can be set and intervals can be conducted for desired durations rather than being limited to physical characteristics of actual terrain.

Generally, as discussed above, a TriDot can measure an individual's swimming, biking and running ability. The calculator 130 can determine a TriDot for these, which in one embodiment can range from one to eighty-five, with one being the lowest and eighty-five being the highest performance level. For example, a TriDot near 85 would generally correspond to the best competitive results of the fastest athletes in each discipline; TriDot's in the mid-range to the performance of average athletes and a TriDot of 25 will generally correspond to competitive results of the slower athletes in competition.

In one embodiment, the computer system 100 can assess relative swimming, biking, and running ability to determine which is the stronger or weaker discipline. Basically, the TriDot values are compared to one another, and whichever has the lowest value is the weakest discipline, and the highest value is the strongest discipline.

The formulas for computing the TriDots for each of swimming, biking, and running used in the invention are derived to obtain normalized values such that, a specific TriDot value for the swim represents the same relative performance ability for the swim as the same TriDot value would represent for bike and run abilities. The range of TriDots is from 1 to 85 with 1 being the lowest ability and 85 being the highest ability. An athlete with the same TriDot value for all three disciplines would have relatively the same performance ability in the swim, bike, and run relative to other swimmers, bikers, and runners. With these relative values known for a given athlete, these abilities can be evaluated against the athlete's race distance (where the proportional distances of the swim, bike, and run legs are different) to determine the optimal training focus. For example, an athlete's swim TriDot may be 10 TriDots lower than his/her bike and run TriDots. If the athlete races at the sprint distance, increasing the swim ability may only improve the overall race time by 1 minute because the swim portion is such a small portion of the race. Even a small improvement on the bike would represent a much bigger benefit because the bike portion is about one half of the total race. However, if the same athlete races the Olympic distance, the swim portion of the race is much more substantial and a swim focus may be advised. Using TriDots allows us to not only measure which disciplines are stronger or weaker, but to quantify by how much. The relative difference between abilities when compared to race distance proportions is a valuable tool for determine training goals and how much priority should be placed on a discipline based on the estimated return on training investment in terms of expected total race time improvement.

Generally, referring to a single TriDot value can be simpler to measure, communicate, and work with than a mass of other performance measures. It gives one a reference point that is in essence the performance result or potential for result that is easier to reference. The TriDots are generally used in conjunction with other athlete data to determine program parameters and produce the training programs.

For example, if a swim TriDot is less than 38 then the swim training needs to be primarily form-focused, working on technique and drills. The lower (“worse”) the swim TriDot value, the more drill work needs to be done. The higher the TriDots the shorter the duration of an athlete's race and the higher race-pace for this athlete will be in relation to their functional threshold. Each athlete's race-pace influences the proportion of overall training volume dedicated to specific intensities. The calculator 130 thus can use the TriDot values to determine the optimum training plan or an individual athlete, through the table look-ups and/or calculations.

As a further example, a low TriDot Olympic distance athlete will be training for approximately a three-hour extensive endurance effort. A high TriDot Olympic distance athlete will be training for a sub-two-hour near-red-line event. These are pure TriDot comparisons, and analysis benefits that provide for clear training program thresholds and influence degrees to which training focus, workloads, intensities, volumes are adjusted. This is an important benefit of the TriDot System that is just guesswork in other systems.

Another entire set of intelligence comes from combinations of TriDot values and athlete characteristics, such as age or body-mass index. For example, an athlete with a medium to high run TriDot AND a very low BMI would need more fitness training, whereas an athlete with a medium to high run TriDot and a very high BMI may be extremely fit but carrying excessive weight. “Fitness” training would produce very little benefit for the athlete in the later scenario, but training that would maintain fitness and alter body composition would be highly effective. Similarly, combining TriDot values with age, training volume, race distance, workload capacity, and so forth provides further information that the system uses to determine the athlete's optimal training plan.

In one embodiment, the calculator 130 can compare the athlete's swimming, biking and running TriDots and show the athlete's relative performance level in each discipline. For example, a TriDot profile of “40-42-35” (swim-bike-run) as calculated by the calculator 130 would indicate that a triathlete's run ability is significantly lower than the triathlete's swim and bike ability. Knowing this, the calculator 130 can choose a training plan to focus the triathlete's training more on improving the athlete's run.

In a further embodiment, the computer system 100 develops training plans based on each of these TriDots. The calculations consist of using the TriDots and other athlete data to look up values, thresholds, ratios, and other training parameters. Thus, TriDots and other athlete data is used to determine how many quality sessions (high-intensity) sessions an athlete should do in a week for each discipline, how much of a particular intensity an athlete should do per week and per quality session, what overall training workload capacity should the program reach prior to the race, based on the projected workload required to complete the race at the anticipated intensity level.

In contrast to the commonly available, off-the-shelf “beginner,” “intermediate” or “advanced” training plans, the computerized system 100 automatically develops an individually customized training plan based upon the individual triathlete's ability and history in each discipline. The computerized system 100 also can shift the focus of a training plan to varying degrees based upon the relative differences between the athlete's swim, bike, and run TriDots.

In one embodiment, analogous to using a TriDot to determine a training intensity, the processor or calculator 130 can use a TriDot to determine a triathlete's optimal pace in racing. For example, the TriCharts of FIG. 4A-4B show expected splits for each discipline. Subsets of these charts can also be generated as needed by the processor 130 for a given triathlete. Based upon the triathlete's various TriDot values, the athlete can use the triathlete's training data and terrain and climate variations for the athlete's specific rate to individual race pace. The system 100 can further generate race pacing tools that allow the athlete to enter the triathlete's TriDots and determine the athlete's ideal pacing strategy.

In a further embodiment of the system 100, the athlete can develop a TriDot profile that compares relative power, stamina, and endurance abilities. By using values based on measurements of a triathlete's power, pace, and/or heart rate data at prescribed intensity levels, the computerized system 100 can reveal relative power, stamina, and endurance abilities. For example, if a triathlete can run a TriDot 40 5k run, but the triathlete's heart rate skyrockets after five miles at their prescribed easy pace, this may indicate that the triathlete's endurance abilities are lacking.

In prior art approaches, triathletes often erroneously think that their training is going well, and only on race day do their “opportunities for improvement” get revealed. However, advantageously, using TriDots such as generated by the computerized system 100 or as revealed in the charts of FIG. 4A and 4B allows the athlete to take a more proactive approach to a triathlete's training and racing without a need to do excessive field tests. The athlete will know throughout the athlete's training where the triathlete needs to improve. And the triathlete will then be ready to compete on race day, with realistic expectations of the performance he or she is capable of.

In one embodiment, assessment options to determine TriDots are as follows. A swimming TriDot value is determined by the processor 130 after either a 10-minute swim or a 800-meter (or yard) trial. A bike TriDot value can be determined by the processor 130 after an 8-minute or 15-mile time trial. A run TriDot value can be determined by the processor 130 after a 12-minute, 5k or 10k time trial.

Generally, prior art VDOT values were based on velocity at VO_{2Max}. However, the extended scale of TriDot values of the system 100 is driven from normalized averages of results of well-trained athletes performing at their Functional Thresholds in each of the disciplines (running, biking and swimming) (which can, for example, be obtained as published splits from public competitions). The TriDot individual performance values for an individual athlete operating at his/her own individual Functional Threshold will be proportional to the reference TriDot values, all normalized to the same scale across each discipline. Different assessments are similarly proportional to standard Functional Threshold values.

For example, if the athlete's Functional Threshold is the power they can sustain for one hour on the bike, then the average power and average heart-rate for someone completing the 15-mile time trial in sixty minutes would be their Functional Threshold Power and Functional Threshold Heart Rate values. If someone completes the 15-mile time trial in forty five minutes, their average power and heart rate will be a specific percent higher than what they could sustain for sixty minutes. The same is true for someone who completes the 15-mile time trial in thirty-five minutes.

In one embodiment, the computation performed by the TriDot system 100 evaluates the time trial results in relation to the fraction of sixty minutes that it took to produce those results and makes the necessary adjustments to the Functional Threshold values which correspond to TriDot values.

In another embodiment, for heart rates, unlike measuring sustained power that is constant for 60 minutes, heart rate lags behind the power being expended. In the initial stages of the time trial the heart rate is lower and gradually climbs. The proportion of the duration of this lower heart rate period is greater the shorter the total time trial. Appropriate adjustments are made in the heart-rate-based training zones.

In a yet further embodiment, TriDots for all three of these disciplines provide values for time trials (often referred to as assessments), threshold pacing, and race-pacing for sprint, Olympic, half iron, and iron distance races. However, training intensities can be handled differently for each discipline other than running. Swim: based on threshold pace for 100 meters (or yards). Bike: based on threshold power if using a power meter; otherwise on heart rate.

The running is based on paces, and exemplary paces for various distances for each intensity level are shown in the Tri-Charts of FIGS. 4A-4B.

TriDot paces (swim—time per 100m; bike—power in watts; run—minutes per mile or kilometer) are approximations based on typical triathletes. Any individual triathlete performance and capabilities could be skewed based on body composition changes physical limitations, and numerous other factors.

Generally, TriDot performance and paces for various distances apply to appropriately trained athletes for the performance and paces for those distances. For example, a run TriDot 50 athlete may be able to run 5k in 19:56 but will not be able to run a 3:10 marathon without proper training for the marathon distance. They are both TriDot 50 performances, but preparation for each is different.

TriDot race splits are based on optimized performances that use race pacing produced by the TriDot System pacing guides, which are generated from TriDot values and which follow a TriDot System generated training program. Generally, terrain, climate, and other conditions can dramatically affect pacing. When possible, it is preferred to conduct assessments and train in conditions similar to those anticipated for race day.

In one embodiment of an employment of the system 100, a triathlete completes a swim, bike and run assessment. The assessment is determining the triathlete's Functional Threshold for each of these three sports. This generates a TriDot score.

Then, the next priority race is determined for the user based upon the TriDot value. This will be explained in more detail below. Then, a training plan request can be generated by the calculator 130 for a next training phase based upon the various TriDot values.

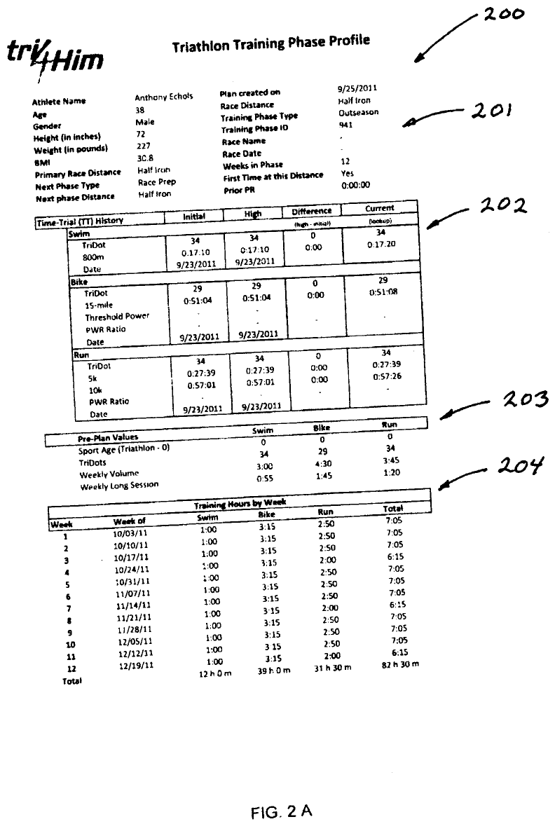

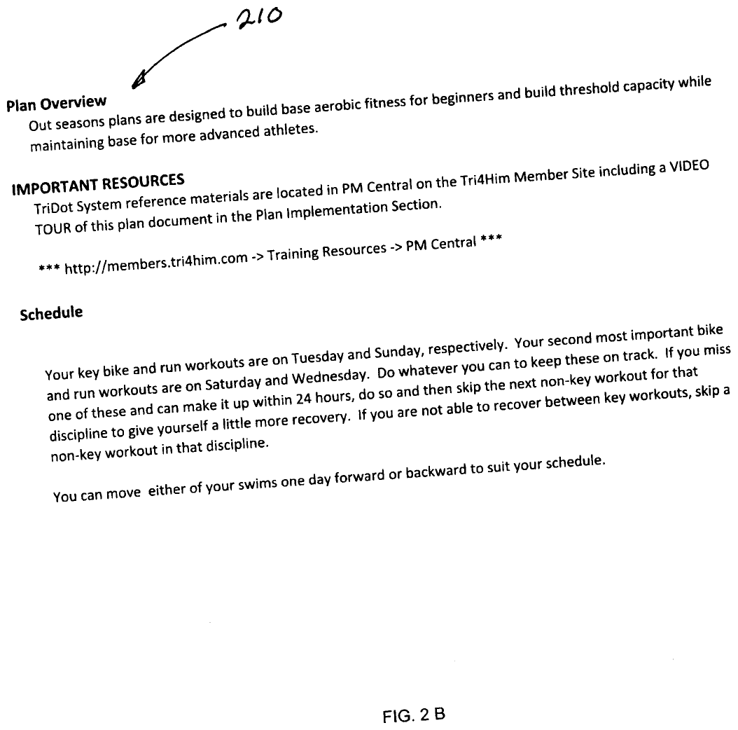

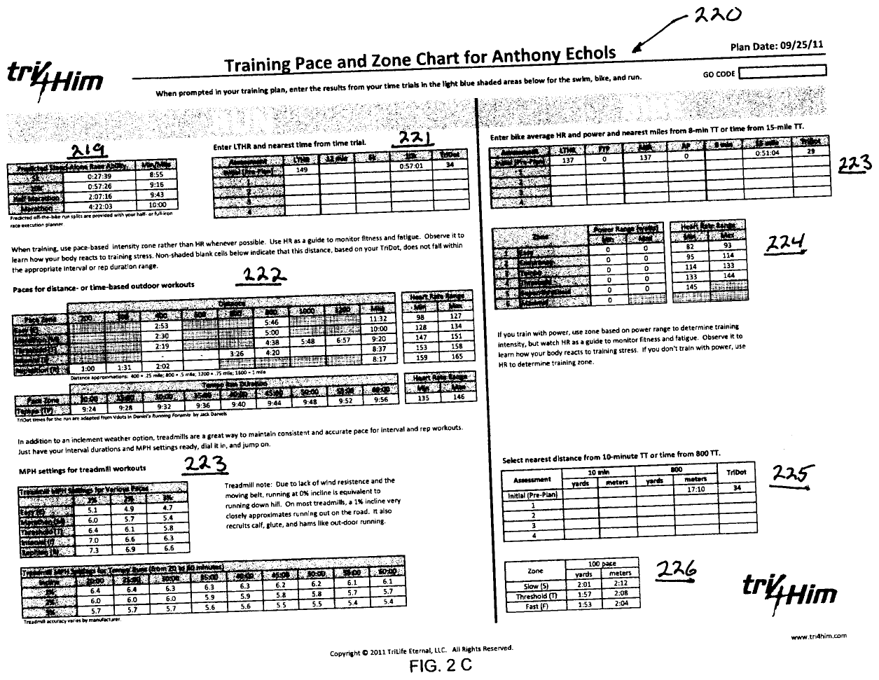

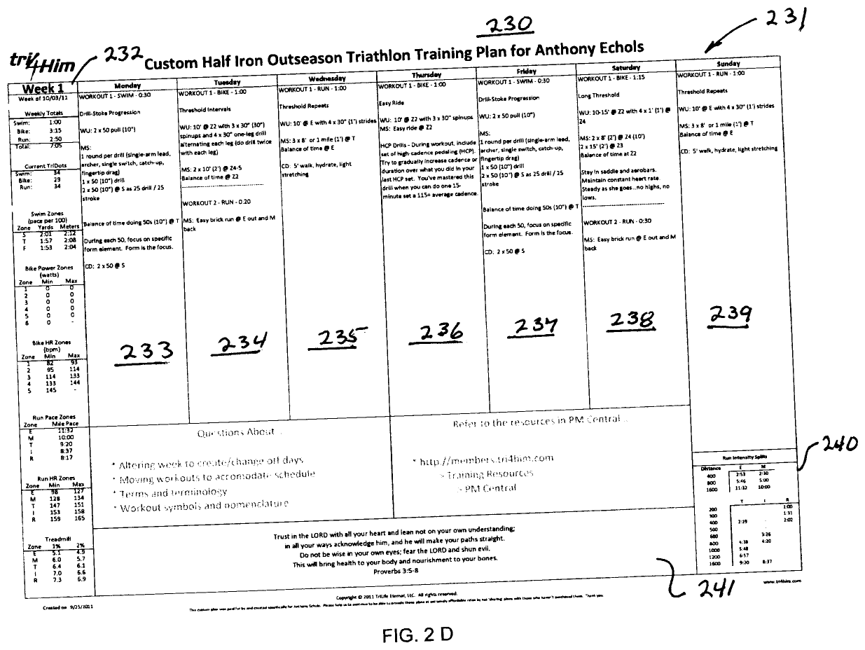

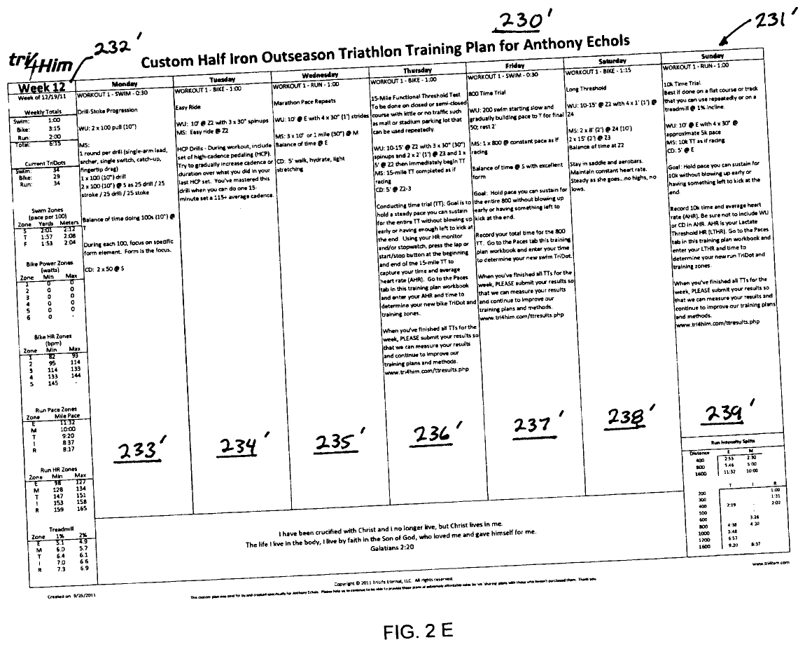

FIG. 2A shows a summary of the the weekly training hours by discipline. FIG. 2B provides training notes accompanying the training plan, some of which notes are standard and some are which are generated by the system by table lookup (such as the days of the week of training), to coordinate to the training plan. FIG. 2C provides the athlete with associated training intensities in terms of power, heart-rate, and pace as appropriate for each discipline, which are generated by the system. For the run, these training intensities are shown in terms of splits for different distances to aid the athlete in conducting training sessions. FIG. 2D provides a detailed, week-by-week training plan generated by the system, which provides detailed daily workouts for all training for each of the 12 weeks long training phase or period. Training zone ranges are listed in the margins for easy reference.

Turning briefly to FIG. 2D, the training plan is generally based on a 2-4 month training phase and is based on the person's TriDot assessments, performance background, race distance, age, sport ages in all three sports, disparity between performance levels to address weaker disciplines, and so on. The training plan for each phase will build on the prior phase and prepare the athlete for the next training to ensure continued growth and long-term development.

In one embodiment, the training plan can include daily workouts with specific objectives, durations, paces, zones, rest duration, and technique pointers. The training plan can be provided on an internet-based implementation of the computerized systems 100 and can include access to a 24/7 on-line source for video or other online media content including articles, guides, and tools. The training plan can include guidance for adjusting a weekly plan to accommodate unexpected changes in the triathlete's routine. The workout plan can include training pace, power and zone by the calculator 130 based on the triathlete's current TriDots from the triathlete's initial assessment and those conducted throughout the triathlete's plan.

In a further embodiment, nutrition and hydration calculators (fueling and cooling) are included, as these are especially important for half and full iron triathletes where hydration should be “spot on.”

The computerized system 100 can further include race execution planning tools that use assessment and training data from key workouts and race rehearsals to determine the athlete's optimal race pacing for half iron man and iron man events. The system 100 can include season planning guidance to help the athlete plan practical and purposeful training plans such as an annual training plan.

Planning guidance generated by the system 100 provides information for the athlete regarding expected starting and ending training volumes for each phase so that the end of one phase will allow for effective transition to the next. For example, the training volume associated with an athlete completing a half iron triathlon would be adequate to transition directly into a full iron distance training phase, whereas a beginner sprint-distance phase would not.

In one embodiment, each phase of training generally lasts between 10 to 20 weeks. There are multiple phases in one year and multiple mesocycles in one phase. Mesocycles are generally 3-4 weeks. These phases derived by the calculator 130 are designed with a fairly consistent weekly pattern, making it easy to plan and coordinate with the rest of the triathlete's life. This pattern is easy to adjust to accommodate unforeseen scheduling conflicts and the need to take days off from training in a way that minimizes loss of fitness. Generally, the structure and spacing of sessions allow for them to be pushed forward or backward a bit, making it easy to adjust the training as necessary.

In a further aspect of an employment of the computerized system 100, lower priority races may be added or changed. Each phase is planned to facilitate progress towards one “A” (most important) race and top priority training goals and developments. Working in lower priority races for fun, to gain race experience, or for any other reason, is easy when using the system 100. Based on the importance of the lower priority race, the athlete can chose to taper and recover or just train through it. The system 100 allows the athlete to make these short-term changes without jeopardizing long-term progress. The plan essentially guides athletes how to go off plan and retain as much of the training benefit as possible, while substituting the race for some of the training.

Turning again to the system 100 of FIG. 1, the computerized system 100 collects, and in some cases measures, comprehensive athletic data and performance assessments, and can normalize it. Normalization involves producing values that correlates the influences swim, bike, and run performance on an equal footing such that they can be compared and used for training program design. The computerized system 100 can analyze the data elements individually and in combination with other elements.

The system 100 can evaluate the results of this analysis against one or more of dozens of standards (thresholds, scales, ratios, and other metric tools.) These metric tools are provided in a program design tool. TriDots, developmental stages, training paces, projected race times, training volumes are stored in a central place. Volume factors, intensity factors, improvement factors, stress factors, stress increase trigger factors, bike/run focus thresholds are located on different computerized matrices, such as spreadsheets, based on race distance. In a further embodiment, the computerized system 100 can move these or make them more presentable if necessary.

The computerized system 100 can determine key program design drivers, and the potentials for improvement addressed above.

The computerized system 100 can establish quantifiable design parameters used to create optimized training.

The computerized system 100 can develop a training program that produces maximum performance gain per training hour and also supports the long-term growth of the triathlete. This is a huge benefit at the training session level. Most, perhaps even all, other training programs prescribe training on a distance basis: e.g., run 5 miles at easy pace or swim 4 repeats of 200 meters at threshold pace. They do this because round distance measures are easy to work with and calculating splits is time consuming and does not appropriately take into consideration physiology. By contrast, however, training responses (physical adaptations) are most effectively stimulated when the athlete works at a specific intensity (relative to Functional Threshold) for a set amount of time relative to the intensity. This is made possible by using the computerized system 100 of the present invention.

Prescribing training that is too long at the prescribed intensity can cause the athlete to reduce their effort in order to complete the prescribed duration, increases risk of injury, and puts unnecessary stress on the athlete beyond what their body is able to absorb. If the prescribed training is too short, it fails to generate the desired training response at all.

The TriDot of the system 100 prescribes training sessions at specific, targeted intensities for specific durations or at distances specifically selected based on knowledge of how long the system determines that the athlete will take to complete the distance. This keeps every interval and repetition highly effective and eliminates unnecessary training stress and injury risk.

The computerized system 100 collects mid-and post-program data to feed back into the system 100. This data is used to adjust and improve system thresholds, factors, algorithms, and other calculations. The feedback data is used by the system to improve itself. The training programs of the system 100 prescribe time trial assessments at key points through the training. In the internet-based embodiment of the computerized system 100, athletes are prompted to enter this data on-line along with their weight, heart-rate data, and so forth. The system 100 also collects actual race results and inputs this into the system and compares this to projected race splits.

The computerized system 100 can use a normalized measurement scale that can serve as a foundation element. In other words, results from swim, bike, and run assessments (appropriate for the triathlete's performance level) correspond to specific TriDot values for each of the three events (bike, swim, run). Generally, For optimum performance, the athlete should train such that he or she raises the lower value TriDot's to be at or near the higher values of the athlete's TriDot values, and then endeavor to raise all TriDot values in a balanced way, such as approximately equally. However, training time limitations may dictate a greater concentration on one or two disciplines to achieve the lowest overall time on race day.

In addition to giving the computerized system 100 the ability to consistently measure and benchmark a triathlete's performance, TriDots provide numerous benefits, including the following:

1) quantification of the degree of performance differences between disciplines providing insight into how much training focus should be shifted to the weaker discipline based on long-term goals and short-term race objectives.

2) establishing specific training paces for various intensity levels for each TriDot. In other words, in order to reach a next TriDot value, a performance goal is also generated by the computerized system 100.

3) making communication of performance levels simple and clear.

4) aiding in determining optimal race splits and pacing. For example: The athlete's bike TriDot value allows us to project the race bike split duration. Based on that duration, we calculate the athlete's exertion level as watts or heart rate as a percent of the athlete's functional threshold. The athlete's run ability is also factored into the bike exertion level as stronger runners can exert a little more on the bike than a weaker runner. Likewise, run split and pacing can be calculated based on the athlete's run TriDot and projected bike split. The longer the bike split, the slower the run split based on the athlete's stand-alone run ability for the same run distance.

5) serving as a basis for developing discipline—specific fitness profiles comparing power, stamina, and endurance abilities.

In addition to using the normalized data from the athlete's performance assessments, each system 100 is designed after analyzing and evaluating 40 athlete-specific data element or more. Athlete-provided data is important; however, the most impactful information is typically that which is derived and developed by the computerized system 100 based upon computations from this data, and key combinations of certain data elements as determined by system 100.

Some of the athlete-provided data includes the following:

Age; gender; body composition (height, weight, BMI); current competitive level; competitive goals; years training or competing in each discipline; developmental state in each discipline; current long sessions for each discipline; current weekly training volume (including overall volume and volume for each discipline); current weekly training load, overall and for each discipline (load is a function of volume and intensity); current performance assessment results (TriDots, times or durations, average heart rates, average power); performance-level differentials (degree of variance) between disciplines; race distance; training phase type and duration; and following training phase type and duration.

When employing the computerized system 100, an efficient way to get faster or go farther is to implement a training program specifically designed for the triathlete.

Turning now to FIG. 2A-2E, illustrated are embodiments of various tabs used in a workbook that is both generated and employed by the computer system 100. In one embodiment, the I/O 110 also includes a printout or spreadsheet embodied in software of a training plan, such as a Profile Tab 210, a Notes Tab 220, a Paces Tab 230, a training plan 240, and a Weekly Tabs 250.

In FIG. 2A, the profile tab 210 displays many of the first-level parameters that were used to develop a plan. It also gives an overview of the weekly values. The data of these profile tabs is partially data submitted by the athlete during the training plan request step. The other parts are generated by the system 100 including developmental stage, weekly volume by discipline. The system 100 can show a plethora of factors and thresholds on this page. This data influences the training program generated by the system 100.

In FIG. 2B, the note tab 220 provides some commentary about the triathlete's plan's focus, structure, or key objectives.

In FIG. 2C, the paces tab 230 of the user interface displayed on a computer display (or printed) has been preloaded with the triathlete's initial assessment values. The assessment data (time trial results) submitted with the plan request are shown here. This includes times or distances, heart rates, average power, etc. The paces tab 230 shows the athlete's training paces and zones for the athlete's swim, bike, and run training. This area will use be used to enter the results of the triathlete's subsequent assessments (time trials) as outlined in the athlete's training plan of FIG. 2D

In FIG. 2D, a training plan 240 is illustrated.

In FIG. 2E, a weekly plan 250 is illustrated.

In one embodiment, the training plan 240 prescribes additional assessments every 3-5 weeks. After each assessment (time trial or other field test), the triathlete is prompted to enter the results on the Paces tab 230. When the athlete enters the triathlete's assessment results, the triathlete's new current TriDots will be updated along with the athlete's training paces and heart-rate zones in the profile tab 210.

Typically, triathletes will progress about 1 TriDot for every 4-6 week period if they strictly adhere to their plan generated by system 100. This progression will typically be slower, however, for triathlete's having a higher performance level and as the performance level is further raised. The triathlete's next plan as expressed in the training plan 240 can typically start with the TriDots that the triathlete finishes up with on his or her previous plan, provided that the athlete can start the athlete's next plan relatively soon thereafter.

In one embodiment, the computerized system 100 has loaded within it in the processor 130 some intermediate TriDot values to allow for the triathlete's performance improvement. Generally, strength and form training are both highly encouraged. However, in one embodiment, because effectively prescribing these requires personal assessments, evaluations, and feedback, they are not specifically included in this system 100 interaction values. To maintain performance, it is generally advisable to continue any strength training that the triathlete is currently doing.

In one embodiment, the plan 240 includes the following:

a) mesocycles. The triathlete plan 230 is structured in four-week mesocycles with three weeks of increasing workload (volume and/or intensity) followed by a week of recovery or assessments. The triathlete should work as hard at recovering on the recovery weeks as the athlete does working during the work weeks.

B) days off. The triathlete plan 230 can use a Monday through Sunday pattern. If the triathlete needs to permanently move a day off, the athlete can adjust the plan to start on a day other than Monday. Moreover, depending on when the triathlete's swim workouts are scheduled, the athlete can move one of the triathlete's swims to a light bike or run day to create an additional day off.

C) bricks (combo workout) It is typically best to perform all workouts as prescribed in the plan. However, it isn't always feasible to perform a scheduled brick workout on the designated day. For example, the triathlete may have time for a bike workout, but not the following short run. Therefore, following are some guidelines for adapting the triathlete's workout when this occurs (not all guidelines may be applicable to a particular plan.)

Include one bike-run brick per week, preferably after the triathlete's longest bike workout. When secondary bricks are scheduled during the week, it is generally for time saving purposes. The run can be completed later in the day. If the athlete cannot complete a brick run, he or she should not complete it later in the day if it could jeopardize a key run the following morning. Race rehearsals done as bricks are key workouts and should ideally always be completed as prescribed.

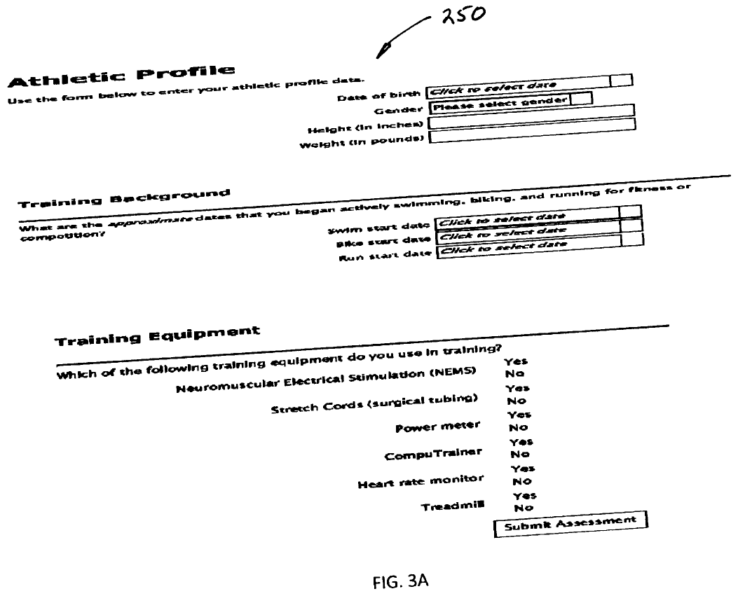

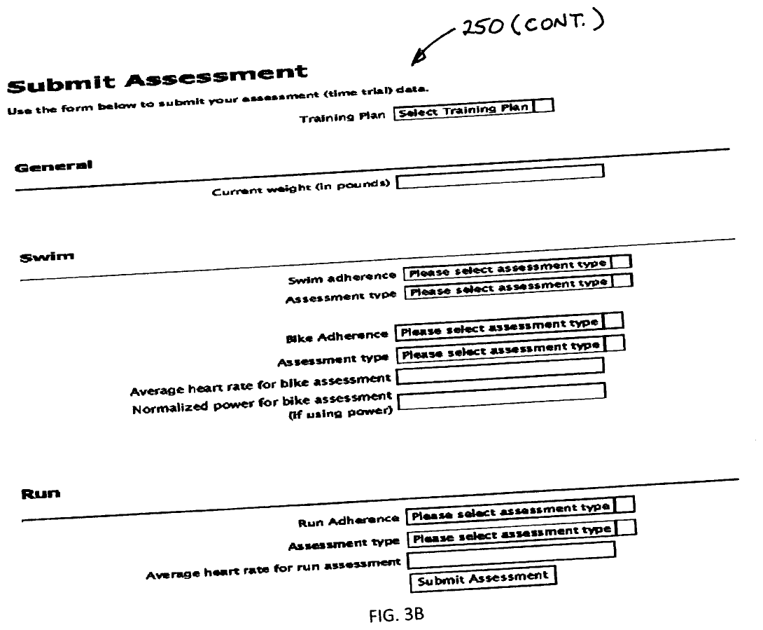

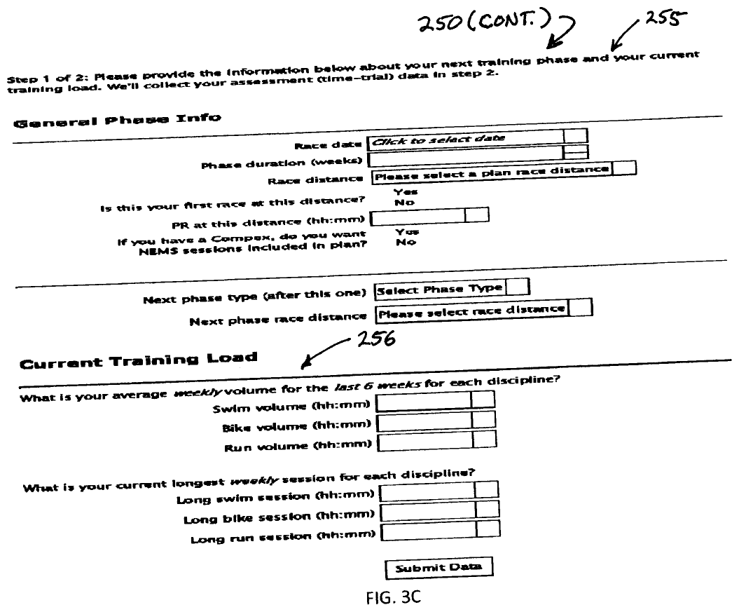

Turning now to FIG. 3A-3C, illustrated are various interfaces that are used by the I/O 110, and also in generating a report.

FIG. 3A is one embodiment of a user interface where the athlete enters much of their static physical characteristics such as gender, height, weight, training background, and so forth. These data are used in conjunction with TriDot values to build training programs. Height and weight are used to calculate BMI and power-to-weight ratios for the bike and run to calculate improvement expectations and training focus based analysis of fitness level and body composition. For the swim, a pace-to-drag ratio can be used. Example: Some athletes will benefit much more from improving body composition (reducing fat) than from developing extreme fitness to carry the excess fat with them on race day. Training background data are used to determine the athlete's developmental stage which is factored into training focus, volume, workload capacity, and so forth. Example: Training workloads (a factor of intensity and volume) above which an athlete is ready for are non-productive and can cause injury. The system uses three developmental stage thresholds based partially on training background to influence the amount and type of training prescribed.

FIG. 3B is one embodiment of a user interface where the athlete enters their time trial assessment data. This happens after the training plan request step. The time trial assessments are specific protocols conducted by the athlete for the swim, bike, and run. Standard assessments are 800 meter (or yard) swim, 15-mile bike, and 5k or 10k run. Completion times, average heart rates, and normalized power output data are entered by the athlete and are used to determine TriDot values. For beginner athletes that are not capable of effectively completing assessments of these distances, the system provides modified beginner protocols for each discipline. These modified distances are a 10-minute swim, an 8-minute bike, and a 12-minute run. The distances traveled by the athlete are used to determine TriDot values. Average heart rate and average normalized power can also be collected to determine training intensity zones. These modified protocols are necessary to assess beginners because 1) they may not have the ability to complete the longer distance and 2) even if they could complete the longer distance, it would be an extensive endurance assessment rather than an intensive endurance or threshold assessment. The standard protocols are appropriate for “trained” athletes and the modified protocols are appropriate for “untrained” athletes. A functional threshold is generally considered the training intensity that an athlete could sustain for about an hour. These modified protocols assess athletes' functional threshold abilities even if they technically cannot even complete a true functional threshold assessment. They allow us to design training for these “untrained” athletes (at shorter durations) that still employ the same highly effective training techniques and principles that the system uses for “trained” athletes capable of longer duration sessions.

FIG. 3C is one embodiment of a user interface where the athlete enters their training request indicating the event type they're training for as well as their current training volume and long sessions by discipline. During the training program design processing, the system compares projected total race time and individual discipline splits to determine training requirements for total volume and long sessions. The athletes' current training volume and long sessions are evaluated against the ultimate requirements and the length of the training phase to calculate the optimal rate of volume and/or long session increases, if necessary.

In one embodiment, the system 100 does not generate any values on these screens of FIG. 3A-3C. These screens instead capture data and load them to a database, which is imported into the system 100.

For best results, the athlete should be conservative when entering data; entering actual data from the athlete's most recent assessments or time trials.

Regarding swim: items (1 and 2) The athlete should enter data from the athlete's most recent functional threshold test Time Trial. These are the same time trial assessments mentioned throughout, such as when discussing FIGS. 3A-3C. This data matches the triathlete's assessment data entered on the pace tab 230 of the triathlete's custom plan 240. If the triathlete is not training and racing with a power meter, the item 2 (FTP) should be left as 0 on the paces tab. FTP is functional threshold power and is only used on the bike. Power is used to measure training and racing intensity similar to using heart rate. Please see sample plan of FIGS. 2A-D. Regarding item 3, select the most appropriate time based on what the triathlete should ride with proper pacing and not with all-out effort. This can be in the plan document on the pace tab.

Item 4 is the triathlete's run performance. The triathlete's ability to run off the bike will partially determine how hard the athlete should run his or her bike split. These values are looked up on a chart or stored as a look-up table in a memory, based upon values previously determined from statistical data based upon many other athletes' collected performance data. Thus, computerized system 100 can obtain these values for use with the individual athlete by a lookup function. The athlete can use TriDot along with the 1-8 performance levels to determine which best describes the athlete's run ability. An example of this can be seen in the scale adjacent to where the athlete enters their values in the pace guide, where Levels 1-8 correlate to ranges of run TriDot values.

The Item 5 section is optional and can be left at all zeroes. However, at the athlete's discretion, the athlete can fine tune the pace guidance by entering values in these fields.

The athlete can increase/decrease watts or heart rate values by entering the corresponding offsets (i.e. enter −5 to decrease watts or to decrease heart rate by 5. Enter 5 to increase watts or heart rate by 5). In making these adjustments, the athlete should consider the recommendations in the pace guide, included in the the provisional application Ser. No. 61/488,084 which is incorporated by reference.

Anticipated race-day temperature, humidity elevation or terrain may warrant lowering the athlete's power guides. Insufficient stamina (decoupling during the later part of the race rehearsals) could leave the athlete to decrease both power and heart rate guides.

Personal experience. The standard guides are based on volumes of race results, but each individual is different.

The bike guidance is broken up into 4 ‘gears’ If the athlete is using power, use that as the athlete's primary guide, but back off if one is reaching the athlete's HR (heart rate) cap.

Gear 1; (The term “gears” is used herein figuratively to communicate distinct effort levels). The first gear is to be used during the first portion of the bike leg. It is arguably the most crucial leg of the entire bike race. It is very easy to overexert during the first portion of the bike race without feeling it until it is too late. If the athlete is using power, the athlete should stick to the correct power, which means to give priority to his or her power number, not his or her perceived exertion of being easy. If the athlete is using only a heart rate monitoring, the athlete should try to keep his or her heart rate down.

Gear 2: after the athlete comes out of gear 1, use gear 2 for all flat portions of the race.

Use gear 3 for all long hills trails taking more than 2 minutes to peak.

Use gear 4 for all long hills less than 2 minutes to peak.

Bike Items 1 and 2: The user would enter data from the athlete's most recent functional threshold test (time trial.) This data should match the triathlete's assessment data entered on the Pace tab of a custom plan. This can be seen in the first two input fields on the first tab of the pace guide spreadsheet,

Item 3 as with the bike fine tuning offsets, the athlete may leave these blank. Adjusting them could be desirable for the same reasons outlined in the bike section.

Using the run guidance The run guidance breaks the run leg into three phases. For Phase 1, just like the bike, it is recommended to take it easy, as illustrated in FIGS. 2A-D.

Phase 2, gradually increase pace from beginning to end Phase 3: pick it up at the finish.

### Bike Assessments

Use one of these two assessments to determine a bike TriDot. A majority of athletes should use the 15-mile time trial. An athlete should use the 8-minute time trial only if he or she cannot complete 15 miles or does not have access to a safe and adequate stretch of road. The athlete should try to use the same assessment throughout each training phase. If the athlete is ready to progress from the 8-minute to the 15-mile assessment, do so when requesting the triathlete's next custom training plan 240.

Time Trials

The time trials are to be done on closed or semi-closed course with little or no traffic such as county road or mall or stadium parking lots that can be used periodically for subsequent tests.

Warm up: The athlete should ride 10-15 minutes at an easy pace (zone 2) with 3×30″ spinups and 2×2′(1)@Zone 3 and 1×5′2 Zone 2 then immediately begin the athlete's time trial.
Main Set: The athlete should ride 15 miles at a constant pace as if racing OR ride 8 minutes at a constant pace as if racing.

Cool Down: The athlete should ride 5 minutes easy (Zone 2 or 30.)

Execution Notes

One goal in the time trial is to hold the pace that the athlete can sustain for the entire distance or time without blowing up early or having something left to sprint at the end. Using a heart rate monitor and stopwatch meter (if the triathlete trains with one), press a lap or start/stop button at the beginning and end of the time trial to capture on time or distance, average heart rate (AHR), and average power. If the athlete doesn't train with power, the athlete will only capture the athlete's time or distance and AHR. The athlete should record his or her total time for the 15 mile or the distance covered for the 8-minute test along with AHR and power. These values are entered into FIG. 3B when submitting a request for plan and FIG. 2C for subsequent assessments during each phase.

In one embodiment, if the triathlete is completing the assessment as prescribed in a custom plan of FIG. 2, using the Paces tab 230 to the training plan workbook 240, and enter data through the interface 110 to determine the triathlete's new TriDot and associated training paces. If the athlete is completing this assessment prior to requesting an initial custom plan, the athlete should submit the triathlete's time or distance AHR and average normalized power with his or her plan request.

Functional Threshold Power Test (with Power Meter on Trainer)

In one embodiment, this test is not used for training plan requests. In another embodiment, this test can be used during a training phase to determine Functional Threshold Power if benchmarking 15-mile not important.

Warm Up: Ride 10 to 15 minutes at an easy pace (Zone 2) with 3/30″(30″) spinups and 2×2′(1′)@Zone 3 and 1×5@ Zone 2 then immediately begin the time trial. It is typically important to be consistent with the triathlete's warm up each time the athlete performs this assessment.

Main Set: 2×20′(2′)—Two sets of 20 minutes with 2 minutes of zone 2 between them.

Cool down: Ride 5 minutes easy (Zone 2 or 30.)

One goal is, in the test, to hold a steady pace that the athlete can sustain for each of the 20-minute repetitions.

Record each 20 minute repetition as an interval on the power meter, wherein the average power afterwards is entered on the Paces Tab 240.

Reading power file: Create 42 minute range that includes the two 20-minute repetitions and the 2 minutes of Zone 2. The normalized watts value for this 42-minute range is the triathlete's Functional Threshold Power (FTP). The triathlete's average heart rate (AHR) for this range is the athlete's lactate threshold heart rate (LTHR), which can be interchangeable with Functional Threshold.

Run Assessments

In one embodiment, one of these assessments is used to determine a TriDot. Most athletes should use the 5k time trial. The athlete should use the 12-minute time trial only if the athlete cannot complete a 5k without stopping to walk. The athlete can use the 10k time trial if he or she runs a sub 50-minute 10k, but may prefer the 5k to minimize training stress. Either one will do the job. The athlete should try to use the same assessment throughout each training phase. If the athlete is ready to progress from one time trial to another, the athlete should do so when requesting the next custom training plan.

Time Trials

The time trials are to be done on a flat course or track that the athlete can use repeatedly or on a treadmill at a @1% incline.

Warm Up

Jog or fast walk 10 minutes at an easy pace (#) with 4 repeats lasting about 30 seconds. at your approximate 5k pace. Make sure that one is warmed up, but do not exhaust oneself.

Main St: Run 5k at a constant pace as if racing OR run 10k at a constant pace as if racing OR run 12 minutes at a constant pace as if racing.

Cool down: Jog or fast walk about 5 minutes at an easy pace (E).

Generally, the athlete's goal in the time trial is to hold pace that he or she can sustain for the entire distance or time without blowing up early or having something left to kick at the end. The athlete can uses a HR monitor and stopwatch, and can press the lap or start/stop button at the beginning and the end of the time trial to capture the time or distance and AHR (average heart rate). Then, the athlete can record his or her total time for the 5k or 10k times or his or her distance covered for the 12-minute TT (time trial) along with his or her AHR.

Generally, if the athlete is completing this assessment as prescribed by the system 100, the athlete should go to the paces tab of the train plan notebook and enter data to determine the athlete's new TriDot and associated training paces.

Alternatively, if the athlete is completing this assessment prior to completing the athlete's custom system 100 plan, the athlete should submit time or distance and AHR along with the athlete's plan request.

Swim Assessments

The athlete should use one of these two assessments to determine his or her swim TriDot. Most swimmers should use the 800 (meter or yard) time trial. The athlete should use the 10-minute time trial only if the athlete cannot complete an 800 without stopping. The athlete should try to use the same assessment throughout each training phase. If the athlete is ready to progress from the 10-minute to the 800 assessment, the athlete should do so when requesting the athlete's next custom training plan.

Time Trials for Swimming

Warm Up

The athlete should swim at least 200 meters, starting slow and gradually building pace to his or her threshold pace for the final 50 meters. The athlete should make sure he or she is warmed up, but should not exhaust him or her self. The athlete should rest for about two minutes. If the athlete does not know his or her threshold pace, the athlete should make a reasonable estimate but err on the side of caution.

Main set: The athlete should swim 800 meters or yards at a constant pace as if racing OR swim 10-minutes at a constant pace as if racing.

Generally, the athlete can use either meters or yards. The calculator 130 can operate using either input. However, the athlete will want to be consistent.

Execution notes: one goal in the time trial is to hold the pace that the athlete can sustain for the entire distance or time without blowing up early or having something left to kick at the end. If the triathlete's time is a part of a workout, swim the balance of the allotted time at a slow pace with excellent form. Otherwise, the athlete should cool down with a slow 200. Then, record the athlete's total time for the 800 time trail or the athlete's distance covered for the 10-minute.

Using the Results

If the athlete is completing this assessment as prescribed in the system 100, the athlete should go to the paces tab 230 of the athlete's training plan workbook 240 user interface and enter the athlete's time or distance to determine the triathlete's new TriDot and associated training paces. The calculator 130 accommodates entry for both meters and yards. If the athlete is not completing this assessment prior to requesting an initial custom plan, submit the athlete's time or distance with the athlete's plan request.

Turning now to FIGS. 4A-4C, illustrated is TriDot Data. FIG. 4A is used in season planning to show what TriDot the athlete should be at prior to progressing to a training phase for a longer race distance. FIGS. 4B and FIG. 4C are described below.

In one embodiment, the TriDot chart of FIG. 4A-4Ce may also be calculated by the calculator 130 of FIG. 1, including calculation of specific training paces for various intensity levels. This system lets the athlete know what pace to set for each workout to ensure that the athlete works in the proper zone to stimulate the desired adaptation. Using this pace as the athlete's guide, rather than heart rate alone, helps the athlete to avoid over-exertion early in that triathlete's workout, which would otherwise cause the athlete to fatigue before the workout is complete. This can help take the guesswork out of determining the intensity of a training regime. These paces can be used in conjunction with heart rate date to measure fitness and improvement.

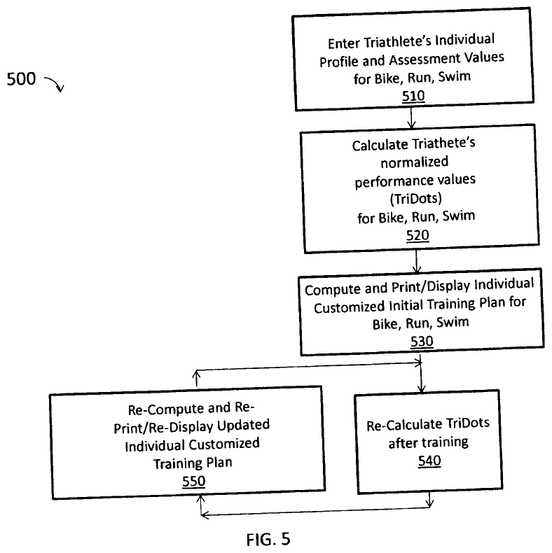

Turning now to FIG. 5, illustrated is a method 500 for calculating TriDots for the disciplines. In further embodiments, the method 500 includes employing the TriDot to generate further exercise regimes.

In a step 510, the triathletes' individual assessment measurement values for various discipline events of a triathlon are entered. These discipline events include running, swimming and biking at one or more distances. These values can be entered into the computerized system 100 and stored in running memory 122, swimming memory 124 and biking memory 126. In a further embodiment, data regarding the triathlete's individual profile values (including, for example, physical parameters such as gender, age, weight, BMI, training age as depicted in FIG. 3B) is also entered, and can be stored in system memory 140.

In a step 520, a TriDot for each of these disciplines (running, biking and swimming) is calculated. These TriDots can be calculated by the system 100 by means of a specially programmed computer processor such as calculator 130.

In a step 530, the TriDots are then used to computer-generate and print and/or display an initial customized training plan for the triathlete. This training plan can be computed and generated by the computerized system 100, and then printed and/or displayed on a computer monitor (not shown).

In a step 540, after training in accordance with the initial training plan, new assessment measurement values for various discipline events of a triathlon are entered (including running, swimming and biking at one or more distances). These new values can be entered into the computerized system 100 and stored in running memory 122, swimming memory 124 and biking memory 126. In a further embodiment, if data regarding the triathlete's individual profile values (including, for example, physical parameters such as gender, age, weight, BMI, training age as depicted in FIG. 3B) has changed, new values can be entered and stored in system memory 140. A new, updated TriDot for each of these disciplines (running, biking and swimming) can then be re-calculated by the system 100 by means of a specially programmed computer processor such as calculator 130.

In a step 550, the re-calculated TriDots can be used to computer-re-generate and re-print and/or re-display an updated customized training plan for the triathlete. This updated training plan can be computed and generated by the computerized system 100. This process of re-calculating TriDots based upon new performance assessments of the individual triathlete following results from training and re-computing and re-printing/re-displaying updated individual customized training plans can be continued indefinitely, as long as the triathlete continues to train and/or otherwise acquire new or different data relevant to computing his/her his/her TriDots and/or individual profile values that are correlative to the computation of the individual customized training plans.

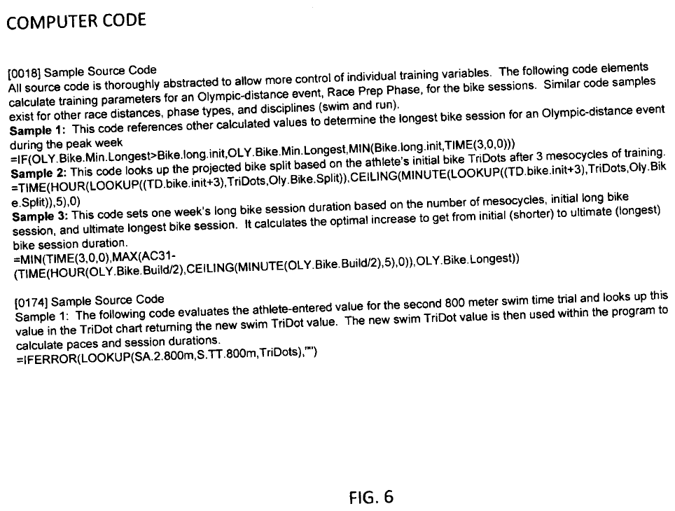

Turning now to FIG. 6, illustrated is representative source code that can be employed by the processor or calculator 110 to determine TriDots.

Generally, the training program is structured by many parameters which are determined based on TriDot values. These include training volume, long sessions, weekly increases, workloads, workload stress; all of these in total and by discipline. Then the design of the training program becomes how does the athlete allocate or focus the training that falls within these parameters. How much intensity per week or per session or per discipline? What type of training responses do we stimulate, how much do we stimulate them, and in what sequence?

These decisions are made by comparing TriDot values and using the scales, ratios, and thresholds derived from TriDot values used in conjunction with other training and athlete data. Examples are the form-focused swim training versus more fitness-focused sessions. Another example would be the progression of how much intensity an athlete can absorb in relative to TriDot value, workload capacity, developmental stage, years training in that discipline, and age. These ratios, scales, and thresholds include numeric values or factors, yes/no triggers, percentages of volume or threshold or workload, or absolute minimums or maximums on aspects of training if data combinations exist.

The following will describe how TriDots are determined by the system in one embodiment.

Introduction

Prior training methods measure abilities from slowest to fastest for a single sport only, such as Jack Daniels VDOT system that gives VDOTs for run values from 30 to 85. Although such VDOT values are known to veteran runners, they do not go below 30 to accommodate beginners and only scale run ability. Unlike such prior single-sport methods, the TriDot training system contains normalized values used to measure abilities of a plurality of disciplines in a relative manner, to optimize overall training and performance for all disciplines at the same time. Additionally, TriDot values associate assessment values to functional threshold abilities and functional threshold abilities to training intensities and paces.

Calculation of TriDots

In one embodiment, the values in the TriDot Chart were calculated in the following manner:

1. A set of triathlon finisher files for many participants for many triathlons, including swim, bike, and run splits for all participants for several different distances, such as sprint, Olympic, half iron, and full iron race distances, are collected and stored in a database.

2. The average finishing times for each discipline and for each race distance against others in the same discipline and distance is calculated. Then, a terrain and climate factor are applied to each race to eliminate finish time differences due to differences in elevation changes and weather, preferably using flat terrain and ideal weather as a baseline comparison.

For example, if the average race time for 5 miles on the flat, at 70 degrees Fahrenheit, is 30 minutes, and the average race time for 5 miles on the flat, at 90 degrees Fahrenheit, is 40 minutes, 10 minutes would be deducted from the average race time for 5 miles on the flat, at 90 degrees Fahrenheit. Where, for example, comparison of several average race times at different temperatures at the same distance shows that the correlation for temperature to time relationship is approximately linear, for intermediate temperatures, a temperature compensation factor can be determined that could be applied to create predicted times for the same distance at any temperature. In such a case, for example, for a 5 miles distance, adding 0.5 minutes per degree Fahrenheit above 70 degrees Fahrenheit and/or subtracting 0.5 minutes per degree Fahrenheit below 70 degrees Fahrenheit (down to some minimum temperature). If the relationship proves to be non-linear, an appropriate non-linear factor (using, for example, an exponential formula or a parabolic formula) may be applied. The same approach can then be used to compensate for terrain.

Once the data is analyzed and compensating factors (linear or non-linear, as appropriate) determined for terrain and temperature, the factors can be applied to times for various terrain and temperature conditions, permitting averaging data from various terrain and temperature conditions together on a normalized basis and allowing prediction of the degree of change that correlates to a given change of temperature and/or terrain from the baseline condition.

3. After applying the terrain and climate factors, the same analytical approach is applied to analyzing finishing times across all races, calculated using one or more of the fastest, slowest, mean, and median finishing times for the swim, bike, and run at each race distance. In the same way as for determination of the temperature and terrain factors as discussed above, a factor to compensate for times for different distances (say 3 miles vs. 5 miles for a run) to allow comparison of runs at various distances, the temperature and terrain being compensated for as discussed above. This is repeated for each discipline (i.e. swim, bike, and run).

4. These values became the top, middle, and bottom parameters for the determination of the TriDot values for the swim, bike, and run race splits, as compensated for temperature, terrain and distance in each discipline. In one example, the top 10 finishing times in each discipline, as compensated for as described above, are averaged and assigned a numerical performance value or TriDot, which, in one example can be 73. In one example, the finishing times for the top 25% of participants can be used as a reference value, and assigned a value, which in one example can be 51. In another example mean finishing times for participants can be used as a reference value, and assigned a value, which in one example can be 39. In one embodiment, where the functional threshold of the athletes whose race times are used as described above to create reference TriDot values have also been separately measured by an independent functional assessment test (such as one conducted on a treadmill, a stationary bike having a power meter, a standalone 800 meter swim etc.), the known athlete's known functional threshold can be compared to the known athlete's performance under race conditions to improve the accuracy and repeatability of the scaling of the reference performance values as compared to functional threshold, the goal being to create a set of relatively stable reference performance values that are based upon readily available data for many well-trained athletes performing at or near their maximum workload capacity. The reference performance values will be relatively stable because the average race times and/or functional threshold assessments for a large number of well-trained athlete's performing at or near their maximum workload capacity changes very little over time.

5. In addition, a compensating factor to compensate for how much slower an off-the-bike run ability is to a stand-alone run ability at the same distance can also be determined, such that run split values would correspond to VDOT run values for the 30 to 85 range. The same approach can be followed to compensate for the slower times expected for an out-of-the water bike ride vs. a stand-alone bike ride.

6. The system then normalizes and aligns comparable bike and swim split values to this same scale as for the run times, thus creating TriDot values for the scale, such that measured equivalent abilities (as compensated for temperature, terrain, distance etc.) for each discipline will yield the same TriDot value.

7. To develop incremental race split values between the bottom, middle, and top values, one or more curve-fitting algorithms (such as available in Microsoft Excel, for example) are applied to the data to determine appropriate formulae relating the degree of training effort represented by an increase in TriDot value. In general, the degree of effort is not a linear relationship, because, for a given training effort, time increases at the bottom of the scale for each higher TriDot are larger than each higher TriDot toward the top of the scale. Qualitatively, this is because of the well-known fact that the faster you get, the harder it is to get even faster. The application of the algorithms to the data by the system, however, permit quantitative determination of one or more mathematical formulas (that are, in general, non-linear) that relate a unit of training effort to a change in a unit change of TriDot values, across the TriDot scale, from the lowest to the highest.

8. Functional threshold paces were calculated for each race split value. An athlete's functional threshold is the intensity of effort that he/she can sustain for an hour. Based on the duration of each race split for each TriDot value, the associated functional threshold pace was calculated using industry standard percentages. For example: The pace to produce a one-hour race split would be approximately the same as the associated functional threshold pace. The longer the race split duration, the lower percentage of the athlete's functional threshold pace.

9. Swim, bike, and run assessment results (distances and times) associated with various functional thresholds were associated with the appropriate TriDot value according to functional threshold pace. See Calculations of Functional Thresholds from Assessments, discussed below.

10. In general, the foregoing TriDot values and formula are based upon data applicable to reasonably well-trained individuals, since most of the available data is for races involving relatively well-trained individuals. However, in the present invention, the same approach to determining formulas for comparing training effort for TriDot values of between 30 and 85, as discussed above, can be applied to the extreme low end swim, bike, and run abilities estimated from very slow untrained individuals. This allows extension of TriDot values and associated performance levels from 30 down to 0, i.e., which may applied to an untrained individual just starting a training program.

Calculation of Phase Training Variables to Maximize Return on Training Investment Based on Current TriDots (Ability), Race Distances, and Time Available to Train

The legs of multisport races vary disproportionately by distance. For example, the swim portion of an Olympic distance triathlon is almost as long as the swim portion of a half iron distance triathlon; however, the bike portion of a half iron race is more than twice as long as an Olympic. Athletes have limited amount of time and energy to train and limited ability to absorb training stress and adapt.

The TriDot System measures the athlete's current ability in each discipline and potential to improve in that discipline and evaluates these relative to the athlete's specified race distance. It calculates the training parameters and variables (including training workload, volume, frequency, sequence, and so forth) for the training phase and sub-periods within the training phase for each discipline.

1. The TriDot System uses the athlete's current TriDots as a measure of current ability in each discipline.

2. These current TriDots are used to produce projected current race splits at the athlete's specific race distance, by applying the formulas determined above that allow comparison across different race distances and under different conditions. For example, if an individual has a TriDot of 50, the formula could predict a time of 30 minutes for a 5-mile run and a time of 37 minutes for a 6-mile run, even if the individual has never run either 5 miles or 6 miles.

3. Improvement factors are used to determine how much an athlete is likely to improve during the training phase to produce an improvement potential. Individual athlete factors include the following: age, gender, years training in each discipline, current training volume, current training workload, BMI, weight, and so forth.

4. The training phase duration is typically 3 months. A phase duration factor is applied for shorter or longer phases.

5. The system database contains known values for improvement for athletes using the training system and for which these factors are known. Each of these factors is based on known results and the athlete's actual data increase or decrease the improvement potential.

For example, assume that, during past training, an athlete having a run TriDot of 30 previously increased his/her TriDot for the run by 1 for a volume of 100 minutes at the athlete's maximum safe (i.e., low injury risk) intensity which, for example, may be an intensity factor of 1.2. This would yield a workload of 120 (i.e., 1.2 intensity factor times 100 minutes). The generally non-linear relationship between improvement and workload input as a function of TriDot values (that has been determined based upon statistical analysis of a large amount of data from many athletes) is then applied to the individual athlete's actual values to yield a predicted curve of the individual athlete's increase in performance as the athlete's TriDot values increase from training. That is, if an average run TriDot 30 athlete will improve 1 TriDot for the run for a workload of 100, and that an average run TriDot 31 athlete will increase 0.9 TriDots for a workload of 100, and this individual athlete's prior history shows that he/she requires a workload of 120 when he/she is at a run TriDot of 30 to increase 1 TriDot to 31, then this individual athlete's required workload for a 1 TriDot run increase from 30 to 31 is 1.2 times as much as the average athlete. Therefore, the system will predict that, when this athlete improves his/her run TriDot to 31, that this athlete must also expend 1.2 times as much workload as an average athlete to increase from a run TriDot of 31 to 32, and so on. Of course, if no athlete-specific data on the amount of TriDot improvement per amount of workload is available (because the athlete has just started training, for example), the system can use the values for average athletes instead. These factors can be determined by the system and entered into a matrix, computer algorithm or spreadsheet, such as the Microsoft Excel spreadsheet included in the Provisional Patent Application, Ser. No. 61/488,084, filed May 19, 2011, the contents of which is hereby expressly incorporated by reference.

6. The improvement potentials are further adjusted based on the athlete's available time to train each week if this time to train falls below specific thresholds based on race distance. For example, suppose that the athlete in the above example does not have 100 minutes of training time per week, and instead only has 80 minutes to train. Although, for a shorter time period, intensity can be increased somewhat to compensate for the reduced amount of training time available, the amount of intensity increase is limited by the potential for injury and the athlete's ability to perform at the higher intensity. If the average athlete, based upon a statistical analysis of a large number of athletes, can safely increase intensity by 10% when training for 80 minutes as compared to 100 minutes, then the system will calculate that this individual athlete can safely increase intensity by 10% when training for 80 minutes as compared to 100 minutes, for a workload of 105.6 during the 80 minutes of training (i.e., intensity factor of 1.32 times 80 minutes). Since the athlete's prior history shows that he/she requires 1.2 times as much workload as the average athlete to achieve the same increase in TriDot values, the system will calculate that the run TriDot 30 athlete can improve 0.88 TriDots per week by training at maximum safe intensity for 80 minutes (i.e., intensity factor of 1.32 times 80 minutes) and, when the athlete reaches a run TriDot of 31, can improve 0.792 TriDots per week for the same 80 minutes at maximum safe intensity factor of 1.32 until he/she reaches a run TriDot of 32, and so on.

7. The algorithms relating improvement potentials by application of various forms of training effort for each of the disciplines (using the normalized values of the TriDots using the mathematical approach discussed above), are applied to determine the change in TriDot values achieved by one or more forms of training effort, to determine potential improvement in race splits at the end of the training phase.

8. The difference between the potential and current race splits for each discipline are compared to determine in which discipline would the potential improvement be most beneficial in overall time savings and by how much.

9. Using the algorithms and methodology discussed above, the system can determining the maximum training workload per week that can be safely performed by the athlete and the amount of predicted improvement in race time for each discipline at the conclusion of the training phase. Based on the overall time savings in each discipline and the degree of difference between them, the system allocates the program training parameters such as intensity, workload, volume, frequency, sequence, and so forth for the training phase and sub-periods within the phase, permitting optimization of the training effort to yield the best overall result for a particular degree of training effort, applied potentially disproportionately to one or more of the run, swim or bike disciplines. For example, if the system calculates that an athlete can decrease his/her overall triathlon time by 3 minutes by spending all his/her available training time on running, can decrease overall triathlon time by 4 minutes by spending all available training time on swimming, and can decrease overall triathlon time by 5 minutes by spending 50% of training time on swimming and 50% of training time on the bike, the system would determine that the third alternative allocation of training time was optimum, because it results in the most improvement in race times for the same number of minutes of training time.

Calculation of Longest Session and Long Session Progression for each Discipline

1. Enter the athlete's current longest session duration for each discipline.

2. Enter the athlete's current training volume and workload.

3. Enter the athlete's time available to train.

4. Enter the athlete's performance assessment.

5. Based upon the performance assessment, use a lookup table or apply the previously determined algorithms and formulae to determine the corresponding the TriDot value.

6. Determine the number of weeks between the current date and the race date.

7. Using an expected improvement factor based on an individual's current TriDot value and other athlete data such as BMI, age, beginning training volume and workload, and so forth, calculate the expected TriDot value at the end of the training phase.

8. Use the expected TriDot value to determine (using a lookup table or applying the previously determined algorithms and formulae) the athlete's expected race split ability at the end of the training phase.

9. Apply the minimum and maximum longest session factors to the expected race split to determine the min and max ranges for the longest session duration. Min and max longest session factors specify the relative duration of the race split to the longest session. The maximum factor has a hard threshold that cannot be exceeded despite longer expected race splits due to the potential training benefit being outweighed by the recovery cost of an excessively long longest session.

10. Compare the resulting longest session duration to the athlete's time available to train and select the longest possible session duration that does not exceed the athlete's time available to train given the actual longest session duration used.

11. Apply a safe duration weekly increase factor times the number of weeks between phase start and the week of the longest session in the training phase to determine the longest possible duration the longest session can be. For example, if the current longest session is 1:00 and the longest safe duration weekly increase is :10 and there are only 8 weeks until the longest session is to be performed, the longest session can at maximum be 2:20 (1:00+:10×8).

Calculation of Interval Distances within Sessions

Individual efforts within training sessions are often prescribed in easy-to-implement units of distance, especially in swim and run training. For example, swim 4×200 m (swim 4 repeats of 200 meters). However, training adaptations are stimulated not by how much distance is covered during an effort but by how long the athlete sustains the effort at the prescribed intensity. An advanced athlete swimming 400 m may take 1:50 while the same distance may take a beginner 5:00. Not only do these durations vary greatly, but the intensity level possible for each these two scenarios varies. An advanced athlete could swim a 200 at an all-out effort (less than two minutes). Neither the beginner nor the advanced swimmer could maintain that level of intensity for 5:00. Depending on the adaptation desired training needs to be prescribed at specific intensities, for specific durations, and with specific amount of rest between efforts.

The TriDot System calculates training set distances based on the athlete's ability to prescribe effective sets for varying intensity levels.

1. Each training session has a training objective that is achieved by training at a specific training intensity(ies).

2. Based on the training objective and the athlete's current workload capacity, one or more sets are created within a training session.

3. Each set contains 1 or more efforts at a prescribed intensity level for a prescribed distance with a prescribed rest duration in between each.

4. The system uses the athlete's TriDot to calculate his/her pace at the intended training intensity.

5. The system uses this pace to determine the distance the athlete will cover in the desired effort duration.

6. The system rounds this distance, if necessary, to a distance that is practical for implementation. (The distance should not be such that the athlete completes it in the middle of the pool. Or if running on the track, the distance is rounded to the nearest 100 m.)

7. Finally, the training set, of one or more efforts, is produced and expressed in terms of distance with the distance being applicable and productive based on the athlete's ability.

Calculations of Functional Threshold from Assessments

Functional thresholds are typically defined as the highest level of training intensity an athlete can sustain for an hour. This threshold is different for the swim, bike, and run. Determining this threshold by requiring the athlete to perform an all-out effort for one hour is very taxing and impairs the athlete's ability to perform subsequent training. The TriDot System uses modified protocols for determining functional thresholds by reference to performance at training effort sustained for other, generally shorter, times. Some are based on a static distance such as an 800 meter swim, a 15-mile bike time trial, or a 5k or 10k run. Some are based on static duration such as 10-minute swim, 8-minute bike time trial, or 12-minute run.

1. The athlete performs the static-distance or static-duration functional threshold assessment and records data such as total time or distance covered, average heart rate, average watts, athlete body weight, and so forth.

2. This data is entered in the system.

3. The TriDot System takes the average pace, heart rate, and/or watts and applies a duration factor based on how long the assessment took the athlete to complete. The duration factor for an athlete completing the assessment in exactly one hour would be 1. The duration factor for completing the assessment in less than one hour would be less than 1. The duration factor is determined based upon formulae and or algorithms comparing assessments for various times that normalize the time and distance of the less-than-one hour assessment to the equivalent distance for a one hour functional threshold assessment for an average athlete having the same TriDot value for the discipline, based upon the statistical averages of many athletes.

4. The duration factor is different for each assessment.

5. The product of the average pace, heart rate, and/or watts and the duration factor produces the functional threshold pace, heart rate, and/or power. In general, these values will be smaller for duration factors less than zero, because the pace, heart rate, and/or power will be less when, for example, a distance is covered in one hour as compared to covering the same distance in less than one hour.

6. The functional threshold pace, heart rate, and power are used to calculate training intensities required to produce particular improvements.

Calculation of Projected Race Paces and Splits

An athlete's first discipline split is simply based on his/her target pace and the race distance. However, subsequent splits are affected by the duration and intensity of preceding legs of a multisport race. Leg workload refers to the workload expended during a specific leg of a multisport event. Workload is a product of the intensity factor times duration. Intensity levels for endurance events typically range from 67% of functional threshold for longer events to over 100% of functional threshold for very short events. The shorter a race is, the higher the percent of threshold an athlete can exert. The TriDot System calculates the race split of subsequent legs of a multisport race as follows:

1. Standard workloads for each leg of the multisport event can be calculated by multiplying TriDot projected duration for each times the intensity percentage based on functional threshold.

2. Each leg's target intensity level is multiplied by a Preceding Workload Factor and a Succeeding Workload Factor. These factors are based on the amount of workload required for preceding and succeeding legs of the event, respectively.

3. The standard workloads based on projected durations and intensities are adjusted for each leg in the order that they'll be performed, first by normalizing the projected durations and intensities based upon the statistical averages of many athletes. If desired, the standard workloads can then be adjusted again, based upon the individual athlete's maximum available training time, maximum training intensity, and the ratio of the athlete's past TriDot increase in a given discipline for a given workload divided by an average athlete's past TriDot increase in a given discipline for a given workload

4. The result is final workloads, durations, and intensities for each leg of the event that account for work that will be done before and after that leg.

5. The final intensities (power or pace or heart rate) are used to prescribe race pacing for each leg of the event.

Calculation of Minimum Weekly Volume and Workload Required

An athlete's ability to perform on race day at a desired intensity for specific duration is relative and proportional to his/her ability to consistently train and recover from week to week at specific training volumes and workloads (volume×intensity). The TriDot System projects the required workload of the target race and then calculates the weekly volume and workload minimums that must be reached to facilitate and render likely the desired performance. Volume is expressed in minutes of training. Workload is minutes of training multiplied by an Intensity Factor. Intensity Factor of 1.0 represents training at the athlete's functional threshold. Intensity Factors below and above 1.0 represent relative differences in intensity below and above functional threshold intensity, respectively. For example: The workload of a single training session including 10 minutes of very easy running followed by 20 minutes of running at threshold could be evaluated as 10×0.5 plus 20×1.0 for a total workload of 25.

The required volumes (durations), intensities, and workloads overall and for each discipline of a specific target race are calculated.

These requirement data are multiplied by Weekly Volume and Workload Factors to produce weekly minimums for overall and discipline-specific training volumes and workloads.

The athlete's current training volume and workloads can be increased gradually from week to week as much as safely possible, considering other athlete variables such as current TriDot, age, gender, BMI, background, and so forth, to reach these minimums prior to the race. As a baseline, the default values for maximum safe increases in volume and workload increases can be set those found to be generally safe for average athletes having the same TriDot, age and gender as the individual athlete, or to a percentage of those increases, such as 90%, to allow for a margin of error. The system allows these default values to be increased or decreased for a particular athlete, based upon injury history or other factors.

Calculation of Minimum Single-Session Volume and Workload Required

An athlete's ability to perform on race day at a desired intensity for a specific duration is relative and proportional to his/her single-session training volume and workload (volume x intensity factor) capacity. The TriDot System projects the required workload of the target race and then calculates the single-session volume and workload minimums that must be reached to facilitate the desired performance. Volume is expressed in minutes of training. Workload is minutes of training multiplied by an Intensity Factor. Intensity Factor of 1.0 represents training at the athlete's functional threshold. Intensity Factors below and above 1.0 represent relative differences in intensity below and above functional threshold intensity, respectively. For example: The workload of a single training session including 10 minutes of very easy running followed by 20 minutes of running at threshold could be evaluated as 10×0.5 plus 20×1.0 for a total workload of 25.

The required volumes (durations), intensities, and workloads overall and for each discipline of a specific target race, to achieve a particular desired race performance, are calculated; in the manner discussed above.

These requirement data are multiplied by Single-Session Volume and Workload Factors to produce single-session minimums for overall and discipline-specific training volumes and workloads.

As discussed above, the athlete's current single-session training volume and workloads are increased gradually from week to week as much as safely possible, considering other athlete variables such as age, BMI, background, and so forth, to reach these minimums prior to the race.

Calculation of Optimal Training Volume, Intensity, Workload based on Time Available to Train

An athlete's ability to perform on race day is proportional to his/her weekly and single-session volume and workload capacity. Most athletes' performance potential is limited by their time available to train rather than their ability to recover from exceedingly higher volumes and workloads. The TriDot System is able to hold training volume within a maximum limit and vary intensity and workload to produce a training program that produces the greatest improvement given the limited volume. While a specific mix or proportion of intensity to volume would be optimal if volume were not limited, as volume is limited, increasing intensity to produce a higher workload capacity may be desirable.

Enter athlete's maximum time available to train. This data can be entered by week and by discipline.

The minimum requirements for weekly and single-session volumes (durations), intensities, and workloads overall and for each discipline of training program for a specific race are compared to the athlete's maximum time available to train.

If the minimum volume requirements are greater than the athlete's maximum time to train, the system increases the proportion of intensity to produce a higher overall workload capacity. The amount of intensity increase is limited based on training volume, race distance, and athlete data such as age, BMI, background, and so forth.

Session Workload Calculated at Interval Level

Typically in other training systems, when workload or time at a specific intensity is calculated for a training session, the entire session duration is multiplied by an intensity factor for the session's primary or target intensity level. For example: A 45-minute run session might include a 30-minute tempo run (slightly under threshold intensity) and a 5-minute warm up and 10-minute cool down run at easy pace. Other training programs would calculate the workload as follows: 45 minutes multiplied by the temp intensity factor of 0.9 (approximately). The TriDot System calculates session workload accurately to the sub-session interval level as follows

1. The duration in minutes of each interval or sub-session effort is multiplied by the associated intensity factor based on the intensities in relation to the athlete's functional threshold to produce a workload value.

2. The total duration and workload value by intensity level, or training zone, is stored and can be referenced for cumulative measures by week, or month, or phase, or other period.

3. All workloads values for each individual session are summed to produce an accurate workload value for the session based on how each minute of the session's duration was spent with respect to training intensity.

Calculation and Use of Workloads and Workload Stress

The concept or measure of workloads and training stress is generally ignored in most training systems. They merely quantify training in terms of volume such as time spent training or distance covered during training.

Workload generally involves multiplying duration by intensity to account for both time and effort. Training stress involves measuring how stressful a workload is for an athlete. Although some coaches use workload and training stress interchangeably, they are very different. Merely because more work is done by one athlete than another doesn't mean that this work is more stressful on the athlete doing more work. The amount of stress should be based on how much work is done and the workload capacity of the athlete, among other factors. More work is required to stress athletes with higher workload capacities. Additionally, how the workload is performed also impacts how stressful it is on the athlete. For example, 30 minutes at intensity X is more stressful than 3×10 minutes at intensity X with 5 minute recoveries between each 10-minute interval.

In the more “advanced” training programs that attempt to quantify workload or training stress, they use the entire session duration and multiply it by an intensity factor for the session's average intensity level. Examples are Training Stress Scores by TrainingPeaks and Dr. Eric Bannister's heart rate-based training impulse (TRIMPS) measures. Duration is typically measured in minutes. Intensity factors usually have a basis of 1.0 where 1.0 represents either threshold or maximal intensity and higher or lower intensities are assigned intensity factors that fall proportionally above or below 1.0. Intensity Factors in all current workload and stress measures have a linear relationship to heart-rate or power. (For example, a tempo run intensity slightly under threshold could be a 0.9 factor). Example: A 45-minute run session might include a 30-minute tempo run (slightly under threshold intensity) and a 5-minute warm up and 10-minute cool down run at easy pace. Other training programs would calculate the workload as follows: 45 minutes multiplied by the tempo intensity factor of 0.9. They do not account for how the work is performed, the workload capacities of the athlete, differences in scaling intensities for each discipline, increases in stress at constant intensity as duration increases, sub-session efforts such as repeats and intervals, and much more. The TriDot System's calculation and use of workloads and training stress can accomplish all of these.

The TriDot System defines, calculates, and uses work and stress as follows:

Workload is an absolute measure of duration (minutes)×effort (intensity factor). In an embodiment of the present invention, Intensity Factors can be different for each discipline and non-linear in relationship to other intensity measures such as heart rate, pace, or power. Example: If the Intensity Factor for threshold effort is 1.0, the Intensity Factor for 50% and 90% of threshold heart rate or power are not necessarily 0.5 and 0.9, respectively, as with other scales and rating systems.

With the TriDot System, as intensity (heart rate, pace, or power) increases toward (or past) specific benchmark intensity such as threshold, the intensity factor increases at an increasing rate. Unlike other systems, workload isn't calculated by session average intensity and total duration. It is calculated at the sub-session level and aggregated to a session workload total. Workload is measured by repetition, interval, session, week, and other periods for each discipline and by each intensity level to establish Workload Histories and Workload Capacities for each.

TriDot System training and race sessions are prescribed for each athlete with specific durations and intensity levels. A single session can be comprised of one or more parts commonly called sets, intervals, and repetitions.

The duration for each part of a session is multiplied by the Intensity Factor based on the intensity level to produce a Workload value for the session part.

All session parts are totaled to produce a session Workload value.

Workload Stress is a relative measure of how stressful a specific type and volume of effort is to an athlete. It includes duration, intensity, and stress. Stress Factors in accordance with an embodiment can be applied to Workload calculations and based on the athlete's Workload Histories and Workload Capacities and other athlete criteria such as height, weight, BMI, age, gender, and so forth. The result is Workload Stress.

Workload stress for the athlete is a function of the athlete's workload capacity. The same workload is less stressful on an athlete with a higher capacity to do work as measured in the system for the prior week's sessions or prior single-sessions successfully performed.

Unlike Workload where the Intensity Factor is constant for a particular effort level, an athlete's Stress Factor will increase with duration even if the athlete's effort is constant. For example, an athlete performing a 40-minute session at a constant threshold intensity will be stressed far greater in the final few minutes of the session than in the initial few minutes. The Stress Factor is progressive with both intensity and duration, but relative to the athlete's abilities and capacities. During training sessions, the Stress Factor diminishes or resets to an initial value based on recovery during the session. For example, for two 20-minute intervals with 10 minutes' rest between, the Stress Factor would increase during the first 20-minute interval and then be diminished during the 10-minute recovery and then resume at a lower value than the end of the first 20-minute interval and increase again during the second 20-minute interval. Recovery Factors can be used to determine the amount of Stress Factor reduction based on elapsed time.

Stress Increase Triggers are durations at which the Stress Factor for a specific intensity level increases. These Stress Increase Triggers are unique for each intensity level based on physiological factors and are unique for each athlete to account for the athlete's Workload History, Workload Capacity, height, weight, BMI, age, gender, and so forth. For example: When performing at threshold intensity during a bike session, an athlete may have Stress Increase Triggers at 20 minutes, 35 minutes, and 53 minutes. At each of these trigger durations, the Stress Factor is increased for each additional minute of exertion. Stress for the first 20 minutes may be quantified as Duration (20)×Intensity Factor (1.0)×Stress Factor (1.0). Stress for the next 15 minutes may be quantified as Duration (15)×Intensity Factor (1.0)×Stress Factor (1.2). Stress for the next 18 minutes may be quantified as Duration (18)×Intensity Factor (1.0)×Stress Factor (1.4). Stress for exertion beyond the 53 minute trigger may be quantified as Duration (X)×Intensity Factor (1.0)×Stress Factor (1.8). Stress factors need not be only multipliers but can be other forms of calculated increases such as exponents, based upon which non-linear mathematical relationship has the best correlation between the Workload Stress when calculated using a particular mathematical relationship and the athlete's performance on race day. The Stress Factors are determined based upon statistical averages of performance data for many athletes, normalized to the equivalent performance that would be expected for athletes having the same TriDot values in each of the respective disciplines.

The measure or rating of Workload Stress for a session is used to determine if the prescribed workload is sufficient to maintain a previously achieved adaptations, stimulate a new adaptations, exceed the athlete's physiological ability to absorb training (adapt), cause injury, and so forth. Workload Stress is also predictive of how long it will take the athlete to recover from the session. The TriDot System uses Workload Stress to calculate and athlete's time required to recover by multiplying a Recovery Factor by Workload Stress.

The TriDot System uses calculations and measures of Workload, Workload Stress, Workload History, Workload Capacity for multiple intensity levels specific to each discipline to prescribe training. The workloads and stress attributable to specific intensity levels are used as parameters to guide the amount and type of training in each intensity level during a given period for each discipline depending on the target race distance.

Calculation of Power-to-Total-Weight Ratio and other factors

An athlete's power-to-weight ratio is a commonly used industry standard for evaluating an athlete's cycling ability and/or fitness. It's typically calculated by dividing an athlete's threshold power output by his/her weight. This measure allows us to benchmark an athlete's fitness over time. When comparing athletes of similar weight, their power-to-weight ratios are a somewhat reliable way of measuring or predicting performance ability. All else being equal, if two athletes weigh the same, the athlete with the higher power-to-weight ratio is capable of outperforming the other.

However, when power-to-weight ratio is used for things such as comparing athletes of different weight, predicting performance ability, predicting potential for performance gains, and so forth, this measure can become unreliable. This is due to several factors.

A straight power-to-weight ratio doesn't take into account the athletes' bike weight. The power generated by an athlete must propel the athlete and the bike. If two athletes have the same power-to-weight ratio but one weighs 50 pounds more than the other, the impact of the bike's weight will have a much greater negative effect on the lighter athlete than the heavier athlete. All else equal, the heavier athlete will perform better despite having the same power-to-weight ratio as the lighter athlete.

A straight power-to-weight ratio also doesn't take into account the body composition of the athletes. Body composition includes characteristics such as height, weight, BMI, body type, and so forth. For example, if two athletes have the same power-to-weight ratio but significantly different BMIs or body types, the athlete with the higher BMI or larger body type will typically create more drag resistance when riding due to their increased surface area. This athlete will have less performance ability despite having the same power-to-weight ratio. Regarding potential for performance gains, if two athletes both have relatively high power-to-weight ratios and one has a much higher BMI, that athlete with the higher BMI will typically have much more capacity to improve performance by improving body composition (losing weight) than the other athlete has to increase fitness. Furthermore, some very heavy athletes may be at or near their peak potential of fitness with the only realistic performance gain potential being achieved through improved body composition. Using power-to-weight ratios alone ignores all of these factors.

The TriDot System uses an athlete's threshold power in addition to other athlete data including height, weight, BMI, and body type along with other athlete data to measure fitness, ability to perform, and potential for improvement gains over specific time periods.

The TriDot System adds the weight of the athlete's bike to the athlete's weight to compute a more reliable power-to-weight ratio for the bike. The athlete's threshold power is divided by the total weight of the athlete and his/her bike. If the athlete's bike weight is unknown, a default value is used based on the athlete's height (taller athletes will typically have slightly larger and heavier bikes), using statistical averages of bike weights for athletes of different heights.

After this power-to-total-weight ratio is calculated, the system can apply further multiple factors to this figure when computing current performance ability and comparing athletes. These factors are based on characteristics such as height, weight, BMI, and body type, based upon normalized values determined from statistical averages of many athletes. These factors make it possible to more accurately predict performance abilities and outcomes and compare athletes. The result value correlates relatively reliably to the athlete's bike TriDot value.

Additionally when predicting the athletes' potential for performance gains, the system can use the power-to-weight and power-to-total-weight ratios and apply an additional set of factors based on athlete characteristics such as height, weight, BMI, and body type, based upon normalized values determined from statistical averages of many athletes, and then can be further adjusted proportionately for an individual athlete. These factors allow the system to determine what type of training will produce the most significant training and what the potential performance gains are for a given training period. For example, by computing the power-to-weight and power-to-total-weight ratios and factors, as discussed above, the system can determine that one athlete with a certain power-to-weight or power-to-total weight ratio might benefit most or have the most potential performance gains from increasing his/her functional threshold power while another athlete with the same ratios but a higher BMI (for example) might benefit most or have the most potential performance gains from improving body composition (losing fat). In each case, based upon the system's calculation of the power-to-weight and power-to-total-weight ratios and additional factors, the system can automatically prescribe different training for each athlete and compute different rates of improvement based on these additional factors.

Calculation of Pace-to-Weight Ratio and other factors

Similar to the process and calculations above for using an athlete's cycling power-to-weight ratio along with other factors related to the athlete's body composition to prescribe the most effective type of training and the potential performance gains for a give training period, the TriDot System calculates a pace-to-weight ratio for running. This ratio is the athlete's running pace at threshold intensity divided by his/her weight.

Typical training programs only take into account the athletes' actual pace at different intensities without regard for the athlete's other body composition characteristics. Ignoring these factors makes prescribing training for athlete's mere guesswork—non-productive and potentially harmful to the athlete. For example: Most training programs or coaches would prescribe running training for all athletes with a 9:00 per mile threshold pace the same. However, a 9-minute-per-mile athlete with a 1.9 BMI and ectomorph build would be a relatively mediocre athlete from a fitness perspective. This athlete would not benefit much from body composition improvement but would benefit greatly from training such as improving form, increasing strength or power, increasing cardio capacity, and so forth. A different 9-minute-per-mile athlete with a 3.2 BMI and endomorph build would deliver the same race-day performance, however (setting aside the excess weight) this athlete is very fit and is likely not going to see much benefits from the training described above for the smaller athlete. In fact, the training prescribed for the smaller athlete would likely cause great harm to a larger athlete. The larger athlete would benefit greatly from training that preserved current “fitness” but improved his/her body composition.

The TriDot system calculates each athlete's pace-to-weight ratio and then applies factors related to the athlete's other body composition characteristics to determine what type of training to prescribe and the anticipated potential for performance gain during a specific training period, in the same manner as described above for determining power-to-total-weight ratio for the bike, and for determining factors based upon normalized values determined from statistical averages of many athletes. Furthermore, if desired, these factors can be further adjusted proportionately for an individual athlete, based upon the ratio of that athlete's historical increase in TriDot values for the run for a given workload, divided by the average athlete's increase in TriDot values for a given workload.

Although the foregoing has been described with respect to the running pace-to-weight ratio, a similar methodology can be employed for determining pace-to-weight ratio for the swim. However, for the swim, a pace-to-drag ratio can be used.

Process for Producing Plan from Athlete Data

Athlete characteristic data is input into the system and stored in system memory 140, including data such as age, gender, height, weight, BMI, body type, number of years training in each discipline, and so forth.

Immediate next training phase data is input and stored in system memory 140 when the athlete or coach on athlete's behalf requests a new training plan. Training phases are generally between 8 and 20 weeks in duration. Data input includes phase start date, phase end date or race date, phase time (in season or off season), race distances for each discipline, prior best time at this race distance, current long training session durations for each discipline, current weekly training volume for each discipline, average training volume for prior period (such as 6 weeks), maximum hours available week to train, desired weekly training hours or range, attitude toward determining training hours (minimalist, enough to deliver strong effort, whatever it takes to maximize results), and so forth.

Subsequent training phase data is input and stored including as much of the same data for the immediate next training phase as possible. This data is for the training phase that will follow the one being produced currently.

Athlete current performance data is input and stored including assessments for each discipline.

Any data that is already in the system from the athlete's prior training phases need not be entered again or re-calculated.

To the extent possible, all processing and calculations described below are done on a discipline-independent basis, cumulative basis, and combination basis (combining bike and run; or combining bike, run, and weight training or other cross-training).

Current (or initial) abilities are calculated such as TriDot values from current assessments, current workloads for various periods (single session, weekly, weekly average), and factors described elsewhere. If the athlete's prior training phase is already in the system, this data can be used rather than re-calculating. Workloads and training duration measures are calculated and stored at the intensity-zone level. If these values are unknown for a new athlete, default distributions of intensity are used such as 70% of training duration is in zone 2 (easy), 20% of training duration is in zone 3 (moderate), and 10% of training duration is in zone 4 (threshold) or above. All of the above are collectively referred to as “Abilities”.

Phase duration is calculated by subtracting the input phase end date from the phase start date.

Required abilities are calculated. Based on the athlete's current abilities described above, the projected fitness gains based on improvement factors, duration of the phase, end-of-phase required abilities (based on projected time to complete race), beginning-of-next-phase requirements, the end-of-phase required abilities are calculated. These abilities are the greater of the abilities required to perform the end-of-phase race at the desired level or the abilities required to begin the next phase. The increase in abilities required to complete the race at the end of the next phase may necessitate more increases in the current phase that only the end-of-current-phase race would require.

Initial abilities are subtracted from required abilities to determine required increases in all abilities.

The system can then break phase into mesocycles (training blocks) generally three to five weeks long based on the duration of the phase. Five-week mesocycles are used as lead-in mesocycles with taper for longer races. Three- and four-week mesocycles are generally used for the remainder of the phase duration. Mesocycles typically have one week of testing and recovery and remaining weeks are “work” weeks where training workloads can be increased.

“Increasable weeks” are totaled to determine the number of weekly increases possible for workloads, intensities, durations, and other sequential training progressions.

Calculation of Weekly Increases

Based on the athlete's abilities and other factors, weekly increase capacities are calculated for each discipline. Weekly increase capacities are totaled to determine the possible or potential end-of-phase abilities.

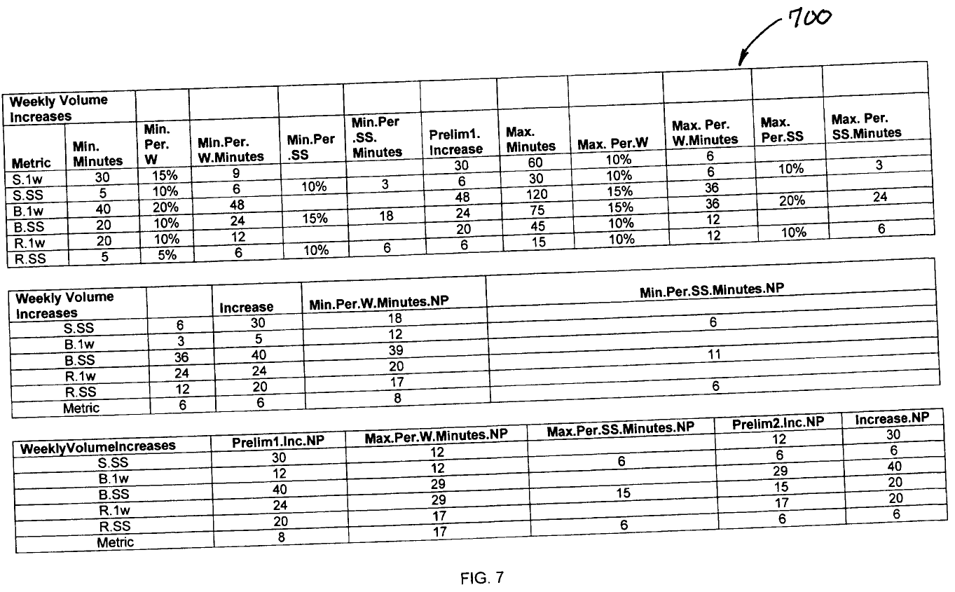

With reference now to FIG. 7, the manner in which weekly increases are calculated will be described. In FIG. 7, the abbreviations are:

Key to Abbreviations:
Metric: S.lw =Swim weekly volume (duration), S.SS =Swim single-session volume, B and R represent Bike and Run
Min.Minutes=Safe weekly minutes that it's always safe to increase from week to week
Min.Per.W=Percent of weekly minutes that it's always safe to increase from week to week
Min.Per.W.Minutes=Safe weekly minutes increase based on Min.Per.W and athlete's actual weekly value
Min.Per.SS=Safe single-session minutes increase as a percent of single session duration
Min.Per.SS.Minutes=Safe single-session minutes increase based on Min.Per.SS and athlete's actual single-session value
Prelim1.Increase=This is the max of Min.Per.W.Minutes and Min.Per.SS.Minutes and represents the amount of increase that is always safe
Max.Minutes=Maximum weekly minutes that it's safe to increase from week to week
Max.Per.W=Maximum percent of weekly minutes that it's safe to increase from week to week
Max.Per.W.Minutes=Maximum safe weekly increase minutes based on Max.Per.W and athlete's actual weekly value
Max.Per.SS=Maximum single-session minutes it's safe to increase as a percent of single session duration
Max.Per.SS.Minutes=Maximum safe single-session minutes increase based on Max.Per.SS and athlete's actual single-session value
Prelim2.Increase=Minimum of Max.Minutes, Max.Per.W.Minutes, and Max.Per. S S .Minutes
Increase=Maximum of Min.Minutes and the minimum of Prelim1.Increase and Prelim2.Increase
Min.Per.W.Minutes.NP=Safe weekly minutes to increase based on Min.Per.W and the athlete's weekly value at the beginning of the Next Phase
Min.Per.SS.Minutes.NP=Safe single-session minutes to increase based on Min.Per.SS and the athlete's single session value at the beginning of the Next Phase
Prelim1.Inc.NP=Maximum of Increase (from prior phase), Min.Per.W.Minutes.NP Max.Per.W.Minutes.NP=Maximum safe weekly increase minutes based on Max.Per.W and the athlete's weekly value at the beginning of the Next Phase
Max.Per.SS.Minutes.NP=Maximum safe single-session minutes increase based on Max.Per.SS and the athlete's single-session value at the beginning of the Next Phase Prelim2.Inc.NP=Minimum of Max.Minutes, Max.Per.W.Minutes.NP, Max.Per. S S .Minutes.NP
Increase.NP=Maximum of the Min.Minutes and the minimum of Preliml.Inc.NP and Prelim2. Inc .N

The table of FIG. 7 is an example of volume (duration) increase capacities. Actual athlete values are not shown but can be calculated from formulas and results shown. Similar tables, calculations, and methods are used for increases in overall workload, workload by discipline, and workload by intensity level, including combinations of these and other measures. Minimum and maximum minute (or workload unit) increases and percentage increases are determined by applying various factors based on athlete characteristics including performance level, age, height, weight, years training in each discipline, and so forth, as previously described. The end-of-phase target abilities including workloads and durations and other related measures are the lesser of the required abilities (based on race or next phase) and the possible abilities (based on increasable potential).

Based on the amount of increase in abilities between beginning and end of phase, training focus, the system chooses an increase strategy and applies increases across mesocycles and weeks of training. For example, if the increases required are nearly equal to capacity, then increases are applied evenly throughout. If minimal increases are required, these increase may be applied based on hours available to train, desired training hours, and other considerations, as determined by the preference input by the user.

Based on the amount of increases required during each mesocycle, weekly training progressions are selected that fit the mesocycle duration, the training focus, and other criteria. For example, if an athlete requires 30 minutes' of increases in his or her single-session long run over a 3-week mesocycle, the system can simply increase the athlete's long run by 10 minutes each week provided that the athlete's increase capacity is 10 minutes or greater. If the athlete's single-session long run is sufficient to meet requirements, but the system determines that a 90-minute increase in weekly duration is needed to meet the total workload required to meet the goal, and at least 30 minutes per week of weekly duration increase is possible, then the system can increase two runs per week by 15 minutes each or add an additional 30-minute run each week. If single-session and weekly volumes already meet requirements, that athlete's workload or workload at a specific intensity can be increased in the same manner.

Workload Stress limits are used in conjunction with workload increase capacities in determining how much increase can be added during a specific period of time or individual session.

The TriDot System's Workload Stress measures are also incredibly valuable for athletes who do not need to increase duration or workload in any way. The TriDot System can introduces Workload Stress in one area while decreasing it in another to stimulate a specific training response, while keeping total Workload Stress within limits that are safe and within limits desired by the user.

Based on weekly training progressions, an athlete's time available to train, preferred training volume, training focus, and other criteria, one or more individual training sessions are constructed for each discipline for each week. Individual sessions are selected from a list of training sessions based on their session type, workload, workload allocation by intensity, target training response, and so forth.

Each session can be uniquely constructed by the system based on the individual athlete's performance abilities and other criteria or characteristics, as described above. Each session in the list of available sessions has a set of instructions for how the session is to be constructed. For example if Bike Session XYZ is selected, it could prescribe the following instructions for constructing the session: Warm Up is 10′ at easy pace; Main set is as many 5-minute intervals at threshold pace that will fit into the athlete's current threshold-pace single-session workload capacity and not exceed 40 minutes with one minute rest between each interval; Cool down is 15′ at easy spin. Instructions can also include relationships between the current session and a similar prior session.

When training is completed, that athlete may manually enter “completed as prescribed” or upload training data from a training device such as a heart-rate monitor or power meter. This data is fed back into system to measure training success and update system thresholds and variables in conjunction with data from other athletes with similar variables.

Individual training session data for an athlete may also be used to dynamically re-calculate future training sessions. For example, if the athlete successfully completes a key bike sessions at prescribed wattage, the system can recognize that the athlete's fitness has improved in increased TriDot value to re-calculate exact current abilities and remainder of phase. Similarly, other indicators (such as lower heart rate, higher wattages, faster paces, quicker recovery for subsequent sessions, and direct athlete feedback) are used to recognize fitness gains and trigger training plan re-calculation for future sessions.

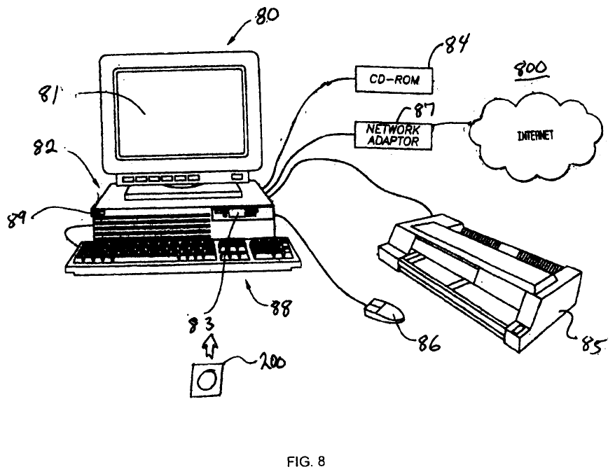

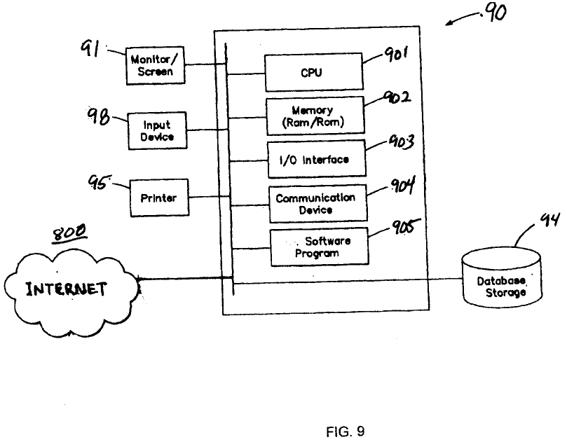

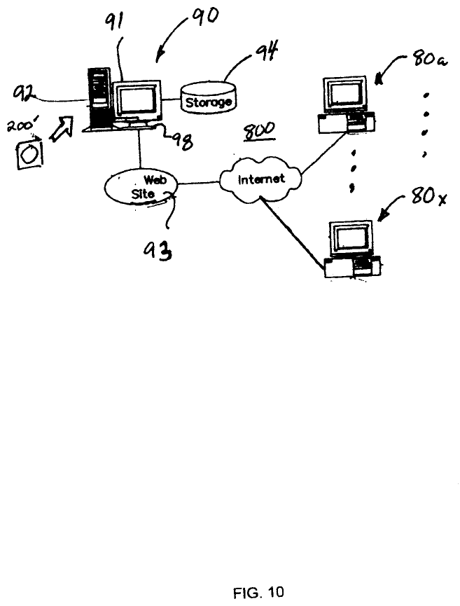

With reference now to FIG. 8-10, the system of the present invention can be implemented on either a local computer system 80 or a central server 90 in communication with one or more computers 80a to 80x over a publically available telecommunication system such as the telephone system or the internet 800. In either case, a user can use a computer terminal 80 as depicted in FIG. 8, or one of computer terminals 80a to 80x (for the internet-based embodiment depicted in FIGS. 9-10.

Computer system 80 includes a display screen or monitor 81, a computer 82 (including a CPU, RAM and/or ROM memory, data storage, communication cards and I/O interfaces), a floppy disk drive 83, a CD-ROM drive 84, a printer 85, a computer mouse 86, a network adapter 87 for communication to the internet 800, a keyboard input device 88, and a USB port 89. Network adapter 87 may be implemented using protocols such as Transmission Control Protocol (TCP) and/or Internet Protocol (IP), well known in the relevant arts. In general, in TCP/IP environments, a IP packet is used as a basic unit of transport, with the source address being set to the TCP/IP address assigned to the source system from which the packet originates and the destination address set to the TCP/IP address of the target system to which the packet is to be eventually delivered.

Where the invention is implemented on a local computer such as computer 80, software 200 on paper, magnetic, optical (such as a DVD or CD), flash memory or other media embodying the steps of the method of the invention would be loaded onto computer 82 by means of floppy drive 83, CD-ROM drive 84, USB port 89, from the internet over network adapter 87. The software 200 can also include data correlating to normalized performance values (i.e. TriDots) in each of the disciplines of interest and for a variety of times, distances and the like, and/or historical results from a large number of athletes in each of the disciplines of interest, permitting computation of the normalized performance values and/or pre-computed look-up tables of the normalized performance values or such data can be loaded onto computer 82 separately. Since the data representing the normalized performance values and/or historical information for a large number of athletes and the mathematical relationships between TriDots and expected performance that are employed by the system to create the customized training plans and to predict competitive performance, though relatively stable, can change over time, it is desirable to periodically re-load updated versions of the software 200 and/or data.

To use the invention, a user (who may be the athlete himself or herself or someone acting on their behalf) would input the athlete's personal information (such as the individual athlete's BMI, prior competition results, etc.) by means of one or more input devices, such as keyboard 88, mouse 86, floppy drive 83 or the like. The personal data input by the user is stored in the computer 82 in either or both of RAM or local storage that also stores the data correlating to the Tri-Dots and mathematical relationships. When instructed to do so by the user, the specially-programed computer 82 implementing the invention will then process the data to create the customized training plans and/or predicted performance results etc., in accordance with the invention. The customized training plans and/or predicted performance results etc. can then be displayed to the user on display screen 81 and/or output in printed form by means of printer 85.

With reference now to FIGS. 9-10, where the invention is implemented on a central server system 90 communicating with one or more local computers 80a to 80x by means of the internet, software 200′ embodying the steps of the method of the invention and data correlating to normalized performance values (i.e. TriDots) in each of the disciplines of interest and for a variety of times, distances and the like, and/or historical results from a large number of athletes in each of the disciplines of interest, permitting computation of the normalized performance values and/or pre-computed look-up tables of the normalized performance values, would be loaded onto the central server system 90. The data representing the normalized performance values for a large number of athletes and the mathematical relationships between TriDots and expected performance that are employed by the system to create the customized training plans and to predict competitive performance, though relatively stable, can change over time. Thus, it is desirable to periodically re-load updated versions of the software 200′ stored on the central server system 90. In the internet-based embodiment of the invention, because users are constantly inputting their own data relating to their performance at competition by means of computer systems 80a to 80x, during assessments and the like, to the central server system 90 over the internet 800. Thus, the system of the invention can be updated to used such new information on a near-real time basis.

In an internet-based embodiment of the invention, to use the invention, a user (who may be the athlete himself or herself or someone acting on their behalf) would use one of computer systems 80a to 80x to access and log-on to permit communication with the central server, after signing up as an authorized user (which may require payment of a fee). The configuration of each of computer systems 80a to 80x would generally be the same as computer system 80 and need not be further described. The log-on information would typically include a user identification number or code and a password.

Once logged-on and authenticated, the user would input the athlete's personal information (such as BMI, prior competition results, etc.) by means of one or more input devices, such as keyboard 88, mouse 86, floppy drive 83 or the like into the user's computer 82. The personal data input by the user can be at least temporarily stored in the user's computer . . . 82 in either or both of RAM or local storage. When instructed to do so by the user, the user's computer 82 will transmit the user's data over the internet 800 to the central server system 90. Central server system 90 includes a specially-programmed computer 92 having data storage 94 loaded with software 200′ implementing the invention, along with the usual monitor 91 and keyboard 98. Computer 92 includes a CPU 901, memory (RAM and/or ROM) 902, an I/O interface 903, a communication device 904 and software 905 (which is the executable version of software 200′).

Central server system 200′ will then process the data to create the customized training plans and/or predicted performance results etc., in accordance with the invention. When requested by a user, central server system 200′, which will commonly be operating as a website 93, will then transmit the customized training plans and/or predicted performance results etc. to the user's computer 82 as a webpage or by other means. The customized training plans and/or predicted performance results etc. can then be displayed to the user on display screen 81 and/or output in printed form by means of printer 85.

### Environment Normalization

### General Description

“Environment Normalization” is the process of adjusting one or more measures of athletic performance to account for environmental factors. Environmental factors fall primarily into two categories: those that are physiological and impact the individual's ability to exert to their potential (such as temperature, humidity, elevation) and those that are physical and limit the results that the individual's exertion produce (such as wind, air density, elevation change (hills), swimming pool format, open-water current). Environment Normalization resolves variances for both categories of factors and provides environment normalized equivalent outcome values (time/duration, power, pace, speed).

Environmental factors have a significant impact on athletic performance. For example, there is less oxygen in the air at higher elevations, and oxygen is required for aerobic activities. Therefore, individuals' aerobic performances are typically diminished at higher elevations. Temperature and humidity affect individuals' ability to dissipate heat to regulate body temperature. The more blood that flows to the skin for cooling means less blood available to carry oxygen and energy to the muscles. The decrease in blood available to transport oxygen and energy will diminish physical performance.

When individuals engage in physical training or competition, they typically do so using a specific target intensity (such as power, pace, speed, or heart rate) or intensity range. These performance metrics are typically determined relative to one or more specific benchmark measures of the individual such as maximum heart rate, VO2max, functional threshold power or pace, and so forth. These benchmarks are determined in the lab, field tests, or training where environmental factors are known.

The intent in training and racing is to perform at a prescribed target intensity to elicit a desired training effect or optimal race outcome. However, the environmental conditions can change from one training/race location to another, can change from one time of day to another, and can change during the training session or race event itself. For example, an individual's target training pace for a specific run might be 7:30/mile if training at 7:00 am in 65 degrees F. and 50% humidity. However, if this same individual were to perform the same session at 3:30 pm in 95 degrees F. and 75% humidity, they might need to slow their pace to 7:43/mile to achieve the same training effect.

Environment Normalization provides individuals with real-time measures of physical exertion intensities relative to their current environmental factors so they can better manage their efforts to achieve optimal or desired outcomes. It can also provide real-time measures of physical exertion intensities for many participants training or competing in disparate environments to allow for apples-to-apples comparisons of progress and outcomes. A specific physical output that results in a performance result of X in one environment will result in a different performance result in another environment. The process of Environment Normalization can hold the intensity constant and determine the adjusted result from one environment to another or it can hold the result constant and determine the adjusted intensity required to achieve that result.

### Example Applications

Pacing Instrument: As individuals train and race, their intensity (power, pace, speed) can be normalized to the current environment. Their training device can adjust their target pace or zone and display the environment normalized metric to the user to help him/her perform at the proper intensity. Coaches and trainers can also use this instrument real-time to monitor their athletes and guide them to optimal performance.

Real-Time Virtual Competition Facilitation (progress and results tracking): Virtual events use environment normalization to produce real-time progress/results for participants in different environments competing in the same event. Individuals could compete against each other in a digital environment on a level playing field by normalizing the environmental effects on their performance. For example, cyclists could login to a virtual environment like Zwift or any number of cycling apps. As they complete in a virtual event, the software could normalize the results from users participating at high altitude versus those a lower altitude (along with wind, temperature, elevation change, and so forth). Likewise, remote runners could all upload gpx files with 13.1-mile route data in different environments. Their results could be environment normalized based on the environmental factors. Environment normalization can also modify each individual's 13.1-mile route making some shorter and some longer to account for environmental differences.

Virtual Race Participant Validation: Virtual events rely on user-provided information such as body weight, bike weight, and aerodynamic drag (CdA) to calculate results. If the user enters false information such as lower body weight, the calculated results of their performance from their actual power generated will be faster than their actual capability. Leveraging the user's historical outdoor training or time trial data and environment normalizing it, environment normalization can validate or certify real-time that performance data submitted during virtual racing events are valid based on the athletes actual environment normalized potential.

### General Process Overview

### Dynamic Pacing Instrument

The individual enters their benchmark performance data and known environment data or the instrument collects this data.

The algorithm can use specific tolerances levels of the individual to certain environmental changes. Example, genetic information, body composition, age, and gender can be used to determine the degree to which increased heat and/or humidity decreases performance. The individual engages in a training session or race with a specified target intensity(ies) such as 250 watts on a bike session to achieve the desired outcome.

As the environmental factors change, the 250 target watch is automatically adjusted up or down depending in real time. The individual's physiological output is held at the desired level because the intensity is changed proportionally with the environment.

### Real-Time Results Tracking for Participants in Different Environments

Participants are registered and connected to a virtual racing platform that tracks activity from training/racing devices such as heart rate monitors, GPS watches, smart trainers, treadmills, bicycle rpm and resistance, swim speed, lap counters, blood oxygen monitors, and so forth.

All individuals participating in a virtual event in different environments connect performance monitors that track metrics such as power, pace, speed, heart rate, and so forth.

All individuals indicate their locations, routes, or otherwise inform the system of their environmental parameters (temperature, humidity, elevation). These data may also come from the performance monitoring devices or location-based weather data feeds.

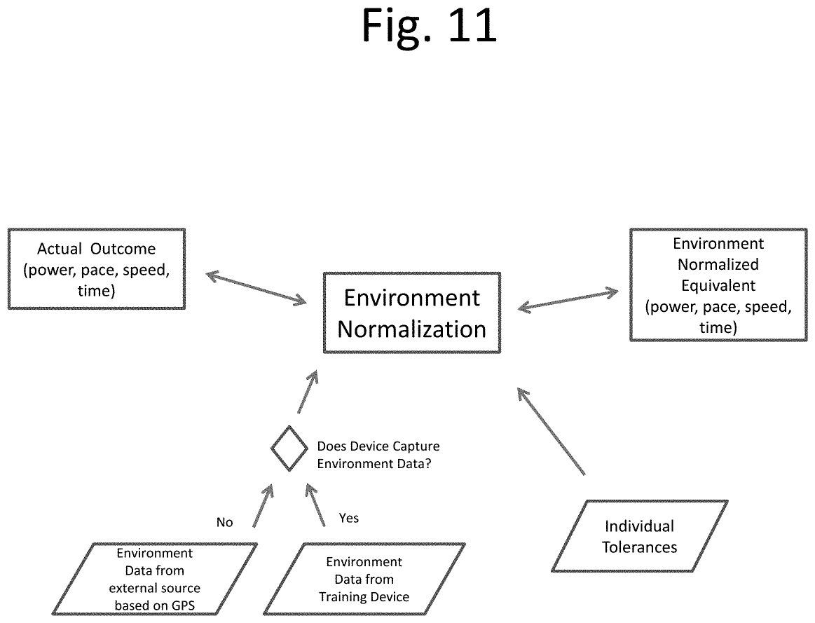

Environment Normalization is used to create a level playing field and display “normalized results” (times, paces, speeds, distances, and so forth) based on the participants performance outputs and environments to simulate the results that would be occurring if all participants were competing in the same environment. This real-time inputs and outputs of this process are illustrated schematically in FIG. 11.

### Calculation Process

Environment Normalization is most simply done by converting actual performance outcomes to/from an Environment Normalized Base value representing what the performance outcome would have been if the individual would have been in the Base Environment of 59° F. with 30% humidity at sea level. Environment Normalization can also be done from one environment to another without converting to/from Environment Normalized Base.

### Calculation Parameters

Acquire Temperature, Humidity, HeatIndex, and Elevation from wearable device, if available, else using GPS coordinates acquire these data from local weather stations and online elevation services.

### Environment Normalized Base (ENB) Parameters

### TempB=15° C./59° F.

### HumidityB=30%

ElevationB=0 meters (sea level)

### AdjustmentFactors (Factors May Vary by up to 10%)

Bike Power HeatFactor=0.002 per degree F.; 0.0036 per degree C.
Bike Power ElevationFactor=0.000068464 per meter above sea level
Run Power HeatFactor==0.003 per degree F.; 0.0054 per degree C.
Run Power ElevationFactor=0.000068464 per meter above sea level
Run Time HeatFactor=0.00154 per degree F.; 0.002772 per degree C.
Run Time ElevationFactor=0.0000124 per foot; 0.000040682 per meter
Swim Time ElevationFactor=0.0000124 per foot; 0.000040682 per meter

### Sample Athlete-Specific Values

Athlete Bike Functional Threshold Power at ENB (Bike FTPB)=300 watts
Athlete Run Functional Threshold Power at ENB (Run FTPB)=250 watts

### Athlete Run Time at ENB (Run TimeB)=1:35:00

Athlete Heat Tolerance (AHT)=1 (typical range of 0.5 to 1.5)
Athlete Elevation Tolerance (AET)=1 (typical range of 0.5 to 1.5)

### Sample Actual Environment Parameters

### TempA=89° F.

### HumidityA=65%

ElevationA=867 meters
If HeatIndex is not provided, calculate it from actual temperature (in F) and humidity.

### HeatIndex=IF (TempA <70, TempA,

### ROUND(−42.379

### +(2.04901523*TempA)

### +(10.14333127*HumidityA)

### −(0.22475541*TempA*HumidityA)

### −(6.83783*(10\hat{ }(−3))*(TempA \hat{ }2))

### −(5.481717*(10\hat{ }(−2))*(HumidityA \hat{ }2))

### +(1.22874*(10\hat{ }(−3))*(TempA \hat{ }2)*HumidityA)

### +(8.5282*(10\hat{ }(−4))*TempA*(HumidityA \hat{ }2))

### −(1.99*(10\hat{ }(−6))*(TempA \hat{ }2)*(HumidityA \hat{ }2)),1))

Calculating Impact on Athletic Performance (calculating from Base Environment to Actual Environment)
Calculate Power Adjustment for Heat (based on temperature and humidity).

### Heat Adjustment for Power=−PowerB*IF(HeatIndexA>TempB, (HeatIndexA−TempB)*AdjustmentFactor, 0)*AHT

It should be noted that AHT and AET are only used in the power calculations below when predicting outcomes or adapting training intensities for an individual athlete. In scenarios where Environment Normalization is used to create a level playing field, these individual tolerances are not used as factors in the adjustments.

### Calculate Power Adjustment for Elevation.

### Elevation Adjustment for Power=−PowerB*ElevationA*AdjustmentFactor*AET

### Calculate Time Adjustments for Heat.

### Heat Adjustment for Time=TimeB*IF(HeatIndexA>TempB, (HeatIndexA−TempB)*AdjustmentFactor , 0)*AHT

Calculate Time Adjustments for Elevation (with Elevation in Feet).
Elevation Adjustment for Time=TimeB*IF(ElevationA>2500 feet, (ElevationA−2,500 feet)*AdjustmentFactor, 0)*AET

### Apply Adjustments

If normalizing Power, apply Heat and Elevation adjustments to base power and re-evaluate time.

EN Power=Power+Heat Adjustment+Elevation Adjustment

EN Power is then used to calculate performance results such as time, pace, speed using the appropriate environmental and other physical parameters (wind speed, wind direction, terrain, and so forth).
If normalizing Time directly, apply Heat and Elevation adjustments to time.

EN Time=Time+Heat Adjustment+Elevation Adjustment

Those skilled in the art to which this application relates will appreciate that other and further additions, deletions, substitutions and modifications may be made to the described embodiments.

What is claimed is:

1. A near real-time GPS and computer-implemented method for measuring the physical capability of an athlete in an athletic endurance event and displaying, in near real-time, performance parameters after adjustment by at least one environmental normalization factor, comprising the steps:
in a specially programmed computer in near real-time, accepting near real-time data input from at least one sensor taken from the group of heart rate monitor for measuring an individual athlete's heart rate and a GPS device for determining distance traveled by the athlete in a time period;
in a specially programmed computer in near real-time, accepting environmental data correlated to said environmental factor in near real-time, said data being accepted from at least one of a remote transmitter or a measurement device worn or carried by the athlete,
in said specially programmed computer, computationally adjusting the athlete's measured actual performance by means of at least one environmental normalization factor taken from the group of temperature, humidity and elevation, wind, air density, and elevation change (hills), to compute an environmentally normalized performance value,
dynamically recalculating said normalized performance values in near real-time based on additional environmental data accepted at subsequent times during said performance,
providing an indication in near real-time to the athlete during said performance regarding whether the current performance is within specified limits as compared to the normalized performance values for the individual athlete including adjustment by said normalized environmental factor to allow the athlete to take timely action during said performance to improve his or her performance.

2. The method of claim 1, wherein said athlete is a first athlete, the method further comprising the steps of:
measuring the physical capability of a second athlete in an athletic endurance event sufficiently similar the athlete endurance event engaged in by said first athlete to permit comparison, displaying, in near real-time, performance parameters after adjustment by at least one environmental normalization factor applicable to the performance of said second athlete, comprising the steps:
In said second specially programmed computer accepting near real-time data input from at least one sensor taken from the group of heart rate monitor for measuring a second athlete's heart rate and a GPS device for determining distance traveled by the second athlete in a time period;
in said specially programmed computer in near real-time, accepting environmental data correlated to said environmental factor applicable to said second athlete in near real-time, said data being accepted from at least one of a remote transmitter or a measurement device worn or carried by a second athlete,
in said second specially programmed computer, computationally adjusting the second athlete's measured actual performance by means of at least one environmental normalization factor taken from the group of temperature, humidity and elevation, wind, air density, and elevation change (hills) in the approximate location of said second athlete, to compute an environmentally normalized performance value for said second athlete,
dynamically recalculating said environmentally normalized performance values for said second athlete in near real-time based on additional environmental data accepted at subsequent times during said performance of said second athlete,
In at least one of said first and second specially programmed computers, comparing the environmentally normalized performance values of each of said first and second athletes and displaying said environmentally normalized performance values for each of said first and second athletes wind, temperatures so that each of said first and second athletes effectively compete on a virtual “level playing field” during the event.

3. The method of claim 1, further comprising the steps of using said environmentally normalized performance values to validate or certify real-time that performance data submitted by said athlete during virtual racing events are valid based on the athlete's actual environmentally normalized potential.
